SEQUENCE LISTING (10 sequences)

SEQ ID NO: 1 - DNA, 4020 bp, Homo sapiens
caccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaaaaaaaaaaaaaaaaaa

SEQ ID NO: 2 - protein, 350 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 3 - DNA, 20 bp, Homo sapiens
tgctggagttggtcggggaa

SEQ ID NO: 4 - DNA, 20 bp, Homo sapiens
tcgtaaggtctccagcgcct

SEQ ID NO: 5 - DNA, 21 bp, Homo sapiens
atggatgatgatatcgccgcg

SEQ ID NO: 6 - DNA, 33 bp, Homo sapiens
ctagaagcatttgcggtggacgatggaggggcc

SEQ ID NO: 7 - DNA, 20 bp, Homo sapiens
ttggctttgtgtccttggcg

SEQ ID NO: 8 - RNA, 21 nt, Homo sapiens
aagaaacgcgguaaucggacu

SEQ ID NO: 9 - RNA, 21 nt, Homo sapiens
aguccgauuaccgcguuucuu

SEQ ID NO: 10 - RNA, 19 nt, Homo sapiens
cuuacgcugaguacuucga

(19) United States
(12) Patent Application Publication
(10) Pub. No.: US 2010/0184820 A1
(43) Pub. Date: Jul. 22, 2010

(54) COMBINATIONS COMPRISING STAUROSPORINES

(76) Inventor: Peter Valent, Vienna (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/305,390

(22) PCT Filed: Jun. 22, 2007
(86) PCT No.: PCT/EP2007/005517

§ 371 Date: Nov. 20, 2009

(30) Foreign Application Priority Data
Jun. 23, 2006 (GB) 0612542.1

(51) Int. Cl. A61K 31/7105; C07H 21/02; C12N 15/74; C12N 5/10; C07H 21/00; A61K 31/711

(52) U.S. Cl. 514/44 A; 536/23.1; 435/320.1; 435/325

(57) ABSTRACT

The present invention relates to a method of treating myelodysplastic syndromes, lymphomas and leukemias, and also solid tumors with a pharmaceutical combination of a FLT-3 kinase inhibitor and an antisense oligonucleotide or a mcl-1-specific RNAi construct. It also relates to the use of a pharmaceutical combination of a histone deacetylase inhibitor and a FLT-3 kinase inhibitor for the treatment of the diseases or malignancies mentioned above and the use of such a pharmaceutical composition for the manufacture of a medicament for the treatment of these diseases or malignancies.

The present invention relates to a method of treating myelodysplastic syndromes, lymphomas and leukemias, in particular Systemic mastocytosis (SM), and also acute myeloid leukemia (AML) and solid tumors such as e.g. colorectal cancer (CRC) and non-small cell lung cancer (NSCLC) with a pharmaceutical combination of a FLT-3 kinase inhibitor and an MCL-1 inhibitor, such as a mcl-1-specific nucleic acid construct. An mcl-1-specific nucleic acid construct includes, but is not limited to an antisense mcl-1 oligonucleotide or a mcl-1-specific RNAi construct. An mcl-1-specific RNAi construct includes but is not limited to a short interfering RNA (siRNA), a micro RNA (miRNA) or a hairpin RNA (shRNA) construct. The invention also relates to the use of a pharmaceutical combination of antisense oligonucleotide or a mcl-1-specific RNAi, and a FLT-3 kinase inhibitor for the treatment of the diseases or malignancies mentioned above and the use of such a pharmaceutical composition for the manufacture of a medicament for the treatment of these diseases or malignancies.

Mastocytosis is a term collectively used for a group of disorders characterized by abnormal accumulation of mast cells (MC) in one or more organ systems. Cutaneous and systemic variants of the disease have been described. Cutaneous mastocytosis (CM) typically develops in early childhood and shows a benign course with frequent spontaneous regression. Systemic mastocytosis (SM) can develop at any age and is characterized by involvement of one or more visceral organs with or without skin involvement. In contrast to CM, SM is a persistent clonal disorder of MC. In a high proportion of cases, the transforming KIT mutation D816V is detectable. In advanced SM, the mutation may also be detectable in other myeloid lineages or even in B lymphocytes. Thus, SM is a disease of multilineage hematopoietic progenitors. The concept that SM arises from a multilineage progenitor is also supported by the notion that these patients can develop an associated hematologic non mast cell lineage disease (AHNMD).

The WHO consensus classification defines four categories of SM: indolent systemic mastocytosis (ISM), mastocytosis with AHNMD (SM-AHNMD), aggressive SM (ASM), and mast cell leukemia (MCL). SM variants differ from each other in their clinical behaviour and prognosis, and usually require different therapies. Notably, in contrast to ISM, patients with ASM or MCL are candidates for treatment with cytoreductive or targeted drugs.

So far, only a few drugs such as interferon-alpha (IFNα) or cladribine (2CdA) have been described to suppress the growth of neoplastic MC in ASM or MCL, and only a few patients show major and durable responses. Some of the novel tyrosine kinase (TK) inhibitors, like such as N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide or nilotinib(4-methyl-3-[[4-(3-pyridinyl)-2-pyrimidinyl]amino]-N-[5-(4-methyl-1H-imidazol-1-yl)-3-(trifluoromethyl)phenyl]benzamide), may also counteract growth of neoplastic MC. Again, however, long lasting responses have yet to be described. Other studies suggest that imatinib inhibits the growth of neoplastic MC in patients with SM. However, the effect of imatinib is only seen in patients in whom neoplastic MC harbour wild type KIT, the KIT mutation F522C, or in patients who have coexistent eosinophilia associated with the FIP1L1/PDGFRα fusion gene. By contrast, imatinib showed little if any effects on neoplastic MC exhibiting KIT D816V. In fact, this KIT mutation, which is detectable in a majority of patients with SM, confers resistance against imatinib.

A number of attempts have recently been made to identify new targets in neoplastic MC in order to develop more effective therapeutic approaches and more effective drug combinations. One promising approach may be to investigate survival-related molecules that are expressed in MC in high grade MC neoplasms (ASM, MCL).

Mcl-1 is a well characterized member of the Bcl-2 family that is considered to act anti-apoptotic in various neoplastic cells. See, e.g., Mendelian Inheritance in Man, MIM accno. 159552. Originally, Mcl-1 was described as a survival-enhancing molecule, expressed during TPA-induced differentiation of leukemic ML-1 cells. Consecutive studies show that Mcl-1 is constitutively expressed in primary neoplastic cells in chronic myeloid leukemia. However, little is known so far about the expression and role of Mcl-1 in other myeloid neoplasms.

It has now been found that FLT-3 kinase inhibitors in combination with an antisense oligonucleotide or a mcl-1-specific RNAi construct possess therapeutic properties. These findings render the combination of a FLT-3 kinase inhibitor and a mcl-1 inhibitor as particularly useful for the treatment myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC).

In particular it has now been found that primary neoplastic MC in all variants of SM including ASM and MCL, as well as the MCL cell line HMC-1, express Mcl-1 in a constitutive manner. In addition, targeting of Mcl-1 in these cells is associated with reduced growth and induction of apoptosis, and with an increased sensitivity to TK inhibitors including N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide, 4-methyl-3-[[4-(3-pyridinyl)-2-pyrimidinyl]amino]-N-[5-(4-methyl-1H-imidazol-1-yl)-3-(trifluoromethyl)phenyl]benzamide, and imatinib.

FLT-3 kinase inhibitors of particular interest for use in the inventive combination are 4-methyl-3-[[4-(3-pyridinyl)-2-pyrimidinyl]amino]-N-[5-(4-methyl-1H-imidazol-1-yl)-3-(trifluoromethyl)phenyl]benzamide, and imatinib and staurosporine derivatives of formula

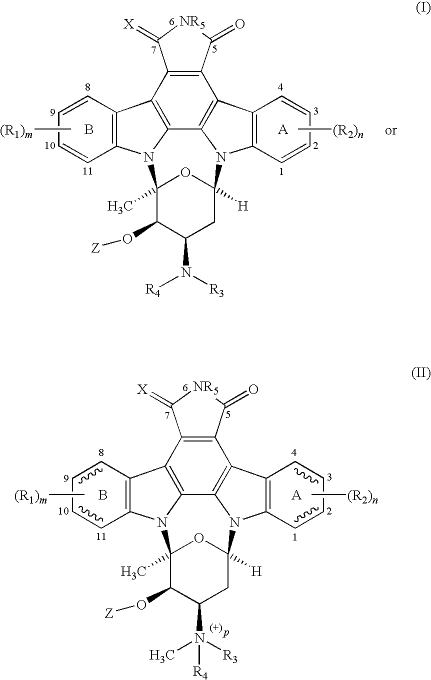

wherein (II) is the partially hydrogenated derivative of compound (I),

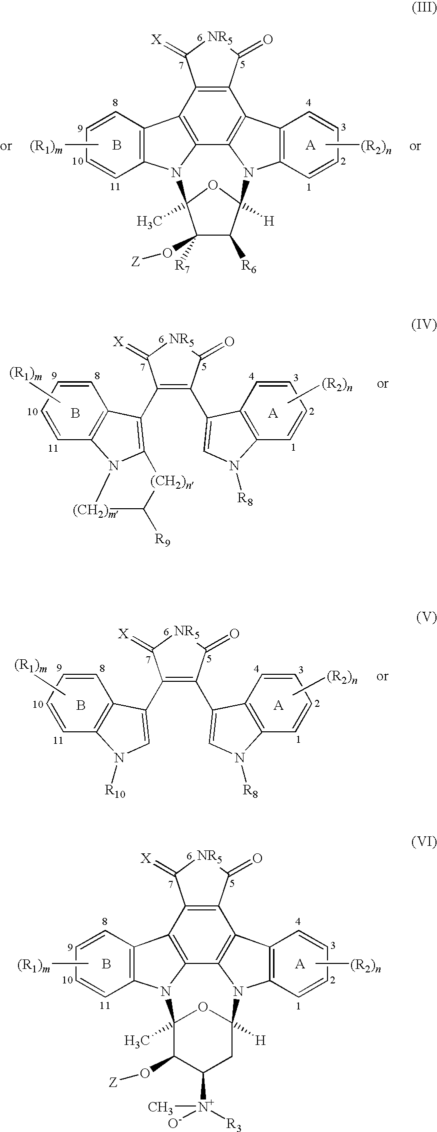

wherein
R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O⁻ acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent;
or if R₃ is acyl with up to 30 carbon atoms, R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy,carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

The general terms and definitions used hereinbefore and hereinafter preferably have the following meanings for the staurosporine derivatives:

The prefix “lower” indicates that the associated radical preferably has up to and including a maximum of 7 carbon atoms, especially up to and including a maximum of 4 carbon atoms.

Lower alkyl is especially methyl, ethyl, n-propyl, isopropyl, n-butyl, isobutyl, sec-butyl, or tert-butyl, and also pentyl, hexyl, or heptyl.

Unsubstituted or substituted alkyl is preferably C₁-C₂₀alkyl, especially lower alkyl, typically methyl, ethyl, n-propyl, isopropyl, n-butyl, isobutyl, sec-butyl, or tert-butyl, which is unsubstituted or substituted especially by halogen, such as fluorine, chlorine, bromine, or iodine, C₆-C₁₄aryl, such as phenyl or naphthyl, hydroxy, etherified hydroxy, such as lower alkoxy, phenyl-lower alkoxy or phenyloxy, esterified hydroxy, such as lower alkanoyloxy or benzoyloxy, amino, mono- or disubstituted amino, such as lower alkylamino, lower alkanoylamino, phenyl-lower alkylamino, N,N-di-lower alkylamino, N,N-di-(phenyl-lower alkyl)amino, cyano, mercapto, substituted mercapto, such as lower alkylthio, carboxy, esterified carboxy, such as lower alkoxycarbonyl, carbamoyl, N-mono- or N,N-disubstituted carbamoyl, such as N-lower alkylcarbamoyl or N,N-di-lower alkylcarbamoyl, sulfo, substituted sulfo, such as lower alkanesulfonyl or lower alkoxysulfonyl, aminosulfonyl or N-mono- or N,N-disubstituted aminosulfonyl, such as N-lower alkylaminosulfonyl or N,N-di-lower alkylaminosulfonyl.

Halogen is preferably fluorine, chlorine, bromine, or iodine, especially fluorine or chlorine.

Etherified hydroxy is especially lower alkoxy, C₆-C₁₄aryloxy, such as phenyloxy, or C₆-C₁₄aryl-lower alkoxy, such as benzyloxy.

Esterified hydroxy is preferably lower alkanoyloxy or C₆-C₁₄arylcarbonyloxy, such as benzoyloxy.

Mono- or disubstituted amino is especially amino monosubstituted or disubstituted by lower alkyl, C₆-C₁₄aryl, C₆-C₁₄aryl-lower alkyl, lower alkanoyl, or C₆-C₁₂arylcarbonyl.

Substituted mercapto is especially lower alkylthio, C₆-C₁₄arylthio, C₆-C₁₄aryl-lower alkylthio, lower alkanoylthio, or C₆-C₁₄aryl-lower alkanoylthio.

Esterified carboxy is especially lower alkoxycarbonyl, C₆-C₁₄aryl-lower alkoxycarbonyl or C₆-C₁₄aryloxycarbonyl.

N-Mono- or N,N-disubstituted carbamoyl is especially carbamoyl N-monosubstituted or N,N-disubstituted by lower alkyl, C₆-C₁₄aryl or C₆-C₁₄aryl-lower alkyl.

Substituted sulfonyl is especially C₆-C₁₄arylsulfonyl, such as toluenesulfonyl, C₆-C₁₄aryl-lower alkanesulfonyl or lower alkanesulfonyl.

N-Mono- or N,N-disubstituted aminosulfonyl is especially aminosulfonyl N-monosubstituted or N,N-disubstituted by lower alkyl, C₆-C₁₄aryl or C₆-C₁₄aryl-lower alkyl.

C₆-C₁₄Aryl is an aryl radical with 6 to 14 carbon atoms in the ring system, such as phenyl, naphthyl, fluorenyl, or indenyl, which is unsubstituted or is substituted especially by halogen, such as fluorine, chlorine, bromine, or iodine, phenyl or naphthyl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkylcarbamoyl, sulfo, lower alkanesulfonyl, lower alkoxysulfonyl, aminosulfonyl, N-lower alkylaminosulfonyl, or N,N-di-lower alkylaminosulfonyl.

The indices n and m are in each case preferably 1, 2 or especially 0. In general, compounds of formula I in which n and m are in each case 0 (zero) are especially preferred.

An aliphatic carbohydrate radical R₃, R₄, R₈ or R₁₀ with up to 29 carbon atoms, which is substituted by acyclic substituents and preferably has a maximum of 18, especially a maximum of 12, and as a rule not more than 7 carbon atoms, may be saturated or unsaturated and is especially an unsubstituted or a straight-chain or branched lower alkyl, lower alkenyl, lower alkadienyl, or lower alkinyl radical substituted by acyclic substituents. Lower alkyl is, for example, methyl, ethyl, n-propyl, isopropyl, n-butyl, isobutyl, sec-butyl or tert-butyl, and also n-pentyl, isopentyl, n-hexyl, isohexyl and n-heptyl; lower alkenyl is, for example, allyl, propenyl, isopropenyl, 2- or 3-methallyl and 2- or 3-butenyl; lower alkadienyl is, for example, 1-penta-2,4-dienyl; lower alkinyl is, for example, propargyl or 2-butinyl. In corresponding unsaturated radicals, the double bond is especially located in a position higher than the α-position in relation to the free valency. Substituents are especially the acyl radicals defined hereinbelow as substituents of R^{o}, preferably free or esterified carboxy, such as carboxy or lower alkoxycarbonyl, cyano or di-lower alkylamino.

A carbocyclic or carbocyclic-aliphatic radical R₃, R₄, R₈ or R₁₀ with up to 29 carbon atoms in each case is especially an aromatic, a cycloaliphatic, a cycloaliphatic-aliphatic, or an aromatic-aliphatic radical which is either present in unsubstituted form or substituted by radicals referred to hereinbelow as substituents of R^{o}. An aromatic radical (aryl radical) R₃ or R₄ is most especially a phenyl, also a naphthyl, such as 1- or 2-naphthyl, a biphenylyl, such as especially 4-biphenylyl, and also an anthryl, fluorenyl and azulenyl, as well as their aromatic analogues with one or more saturated rings, which is either present in unsubstituted form or substituted by radicals referred to hereinbelow as substituents of R^{o}. Preferred aromatic-aliphatic radicals are aryl-lower alkyl- and aryl-lower alkenyl radicals, e.g. phenyl-lower alkyl or phenyl-lower alkenyl with a terminal phenyl radical, such as for example benzyl, phenethyl, 1-, 2-, or 3-phenylpropyl, diphenylmethyl(benzhydryl), trityl, and cinnamyl, and also 1- or 2-naphthylmethyl. Of aryl radicals carrying acyclic radicals, such as lower alkyl, special mention is made of o-, m- and p-tolyl and xylyl radicals with variously situated methyl radicals.

A cycloaliphatic radical R₃, R₄, R₈ or R₁₀ with up to 29 carbon atoms is especially a substituted or preferably unsubstituted mono-, bi-, or polycyclic cycloalkyl-, cycloalkenyl-, or cycloalkadienyl radical. Preference is for radicals with a maximum of 14, especially 12, ring-carbon atoms and 3- to 8-, preferably 5- to 7-, and most especially 6-member rings which can also carry one or more, for example two, aliphatic hydrocarbon radicals, for example those named above, especially the lower alkyl radicals, or other cycloaliphatic radicals as substituents. Preferred substituents are the acyclic substituents named hereinbelow for R^{o}.

A cycloaliphatic-aliphatic radical R₃, R₄, R₈ or R₁₀ with up to 29 carbon atoms is a radical in which an acyclic radical, especially one with a maximum of 7, preferably a maximum of 4 carbon atoms, such as especially methyl, ethyl, and vinyl, carries one or more cycloaliphatic radicals as defined hereinabove. Special mention is made of cycloalkyl-lower alkyl radicals, as well as their analogues which are unsaturated in the ring and/or in the chain, but are non-aromatic, and which carry the ring at the terminal carbon atom of the chain. Preferred substituents are the acyclic substituents named herein below for R^{o}.

Heterocyclic radicals R₃, R₄, R₈ or R₁₀ with up to 20 carbon atoms each and up to 9 heteroatoms each are especially monocyclic, but also bi- or polycyclic, aza-, thia-, oxa-, thiaza-, oxaza-, diaza-, triaza-, or tetrazacyclic radicals of an aromatic character, as well as corresponding heterocyclic radicals of this type which are partly or most especially wholly saturated, these radicals—if need be—possibly carrying further acyclic, carbocyclic, or heterocyclic radicals and/or possibly mono-, di-, or polysubstituted by functional groups, preferably those named hereinabove as substituents of aliphatic hydrocarbon radicals. Most especially they are unsubstituted or substituted monocyclic radicals with a nitrogen, oxygen, or sulfur atom, such as 2-aziridinyl, and especially aromatic radicals of this type, such as pyrryl, for example 2-pyrryl or 3-pyrryl, pyridyl, for example 2-, 3-, or 4-pyridyl, and also thienyl, for example 2- or 3-thienyl, or furyl, for example 2-furyl; analogous bicyclic radicals with an oxygen, sulfur, or nitrogen atom are, for example, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, or benzo-thienyl, typically 2- or 3-benzothienyl; preferred monocyclic and bicyclic radicals with several heteroatoms are, for example, imidazolyl, typically 2- or 4-imidazolyl, pyrimidinyl, typically 2-or 4-pyrimidinyl, oxazolyl, typically 2-oxazolyl, isoxazolyl, typically 3-isoxazolyl, or thiazolyl, typically 2-thiazolyl, and benzimidazolyl, typically 2-benzimidazolyl, benzoxazolyl, typically 2-benzoxazolyl, or quinazolyl, typically 2-quinazolinyl. Appropriate partially or, especially, completely saturated analogous radicals may also be considered, such as 2-tetrahydrofuryl, 2- or 3-pyrrolidinyl, 2-, 3-, or 4-piperidyl, and also 2-or 3-morpholinyl, 2- or 3-thiomorpholinyl, 2-piperazinyl and N-mono- or N,N′-bis-lower alkyl-2-piperazinyl radicals. These radicals may also carry one or more acyclic, carbocyclic, or heterocyclic radicals, especially those mentioned hereinabove. The free valency of the heterocyclic radicals R₃ or R₄ must emanate from one of their carbon atoms. Heterocyclyl may be unsubstituted or substituted by one or more, preferably one or two, of the substituents named hereinbelow for R^{o}.

Heterocyclic-aliphatic radicals R₃, R₄, R₈ or R₁₀ especially lower alkyl radicals, especially with a maximum of 7, preferably a maximum of 4 carbon atoms, for example those named hereinabove, which carry one, two, or more heterocyclic radicals, for example those named in the preceding paragraph, the heterocyclic ring possibly being linked to the aliphatic chain also by one of its nitrogen atoms. A preferred heterocyclic-aliphatic radical R₁ is, for example, imidazol-1-ylmethyl, 4-methylpiperazin-1-ylmethyl, piperazin-1-ylmethyl, 2-(morpholin-4-yl)ethyl and also pyrid-3-ylmethyl. Heterocyclyl may be unsubstituted or substituted by one or more, preferably one or two, of the substituents named hereinbelow for R^{o}.

A heteroaliphatic radical R₃, R₄, R₈ or R₁₀ with up to 20 carbon atoms each and up to 10 heteroatoms each is an aliphatic radical which, instead of one, two, or more carbon atoms, contains identical or different heteroatoms, such as especially oxygen, sulfur, and nitrogen. An especially preferred arrangement of a heteroaliphatic radical R₁ takes the form of oxa-alkyl radicals in which one or more carbon atoms are replaced in a preferably linear alkyl by oxygen atoms preferably separated from one another by several (especially 2) carbon atoms so that they form a repeating group, if need be multi-repeating group (O—CH₂—CH₂—)_{q}, wherein q=1 to 7.

Especially preferred as R₃, R₄, R₈ or R₁₀, apart from acyl, is lower alkyl, particlularly methyl or ethyl; lower alkoxycarbonyl-lower alkyl, especially methoxycarbonylmethyl or 2-(tert-butoxycarbonyl)ethyl; carboxy-lower alkyl, especially carboxymethyl or 2-carboxyethyl; or cyano-lower alkyl, especially 2-cyanoethyl.

An acyl radical R₃, R₄, R₆, R₇, R₈, R₉, or R₁₀with up to 30 carbon atoms derives from a carboxylic acid, functionally modified if need be, an organic sulfonic acid, or a phosphoric acid, such as pyro- or orthophosphoric acid, esterified if need be.

An acyl designated Ac¹ and derived from a carboxylic acid, functionally modified if need be, is especially one of the subformula Y—C(═W)—, wherein W is oxygen, sulfur, or imino and Y is hydrogen, hydrocarbyl R^{o} with up to 29 carbon atoms, hydrocarbyloxy R^{o}—O—, an amino group or a substituted amino group, especially one of the formula R^{o}HN— or R^{o}R^{o}N— (wherein the R^{o} radicals may be identical or different from one another).

The hydrocarbyl (hydrocarbon radical) R^{o} is an acyclic (aliphatic), carbocyclic, or carbocyclic-acyclic hydrocarbon radical, with up to 29 carbon atoms each, especially up to 18, and preferably up to 12 carbon atoms, and is saturated or unsaturated, unsubstituted or substituted. Instead of one, two, or more carbon atoms, it may contain identical or different heteroatoms, such as especially oxygen, sulfur, and nitrogen in the acyclic and/or cyclic part; in the latter case, it is described as a heterocyclic radical (heterocyclyl radical) or a heterocyclic-acyclic radical.

Unsaturated radicals are those, which contain one or more, especially conjugated and/or isolated, multiple bonds (double or triple bonds). The term cyclic radicals includes also aromatic and non-aromatic radicals with conjugated double bonds, for example those wherein at least one 6-member carbocyclic or a 5- to 8-member heterocyclic ring contains the maximum number of non-cumulative double bonds. Carbocyclic radicals, wherein at least one ring is present as a 6-member aromatic ring (i.e. a benzene ring), are defined as aryl radicals.

An acyclic unsubstituted hydrocarbon radical R^{o} is especially a straight-chained or branched lower alkyl-, lower alkenyl-, lower alkadienyl-, or lower alkinyl radical. Lower alkyl R^{o} is, for example, methyl, ethyl, n-propyl, isopropyl, n-butyl, isobutyl, sec-butyl or tert-butyl, and also n-pentyl, isopentyl, n-hexyl, isohexyl and n-heptyl; lower alkenyl is, for example, allyl, propenyl, isopropenyl, 2- or 3-methallyl and 2- or 3-butenyl; lower alkadienyl is, for example, 1-penta-2,4-dienyl; lower alkinyl is, for example, propargyl or 2-butinyl. In corresponding unsaturated radicals, the double bond is especially located in a position higher than the α-position in relation to the free valency.

A carbocyclic hydrocarbon radical R^{o} is especially a mono-, bi-, or polycyclic cycloalkyl-, cycloalkenyl-, or cycloalkadienyl radical, or a corresponding aryl radical. Preference is for radicals with a maximum of 14, especially 12, ring-carbon atoms and 3- to 8-, preferably 5- to 7-, and most especially 6-member rings which can also carry one or more, for example two, acyclic radicals, for example those named above, especially the lower alkyl radicals, or other carbocyclic radicals. Carbocyclic-acyclic radicals are those in which an acyclic radical, especially one with a maximum of 7, preferably a maximum of 4 carbon atoms, such as especially methyl, ethyl and vinyl, carries one or more carbocyclic, if need be aromatic radicals of the above definition. Special mention is made of cycloalkyl-lower and aryl-lower alkyl radicals, as well as their analogues which are unsaturated in the ring and/or chain, and which carry the ring at the terminal carbon atom of the chain.

Cycloalkyl R^{o} has most especially from 3 up to and including 10 carbon atoms and is, for example, cyclopropyl, cyclobutyl, cyclopentyl, cyclohexyl, cycloheptyl, and cyclooctyl, as well as bicyclo[2,2,2]octyl, 2-bicyclo[2,2,1]heptyl, and adamantyl, which may also be substituted by 1, 2, or more, for example lower, alkyl radicals, especially methyl radicals; cycloalkenyl is for example one of the monocyclic cycloalkyl radicals already named which carries a double bond in the 1-, 2-, or 3 position. Cycloalkyl-lower alkyl or -lower alkenyl is for example a -methyl, -1- or -2-ethyl, -1- or -2-vinyl, -1-, -2-, or -3-propyl or -allyl substituted by one of the above-named cycloalkyl radicals, those substituted at the end of the linear chain being preferred.

An aryl radical R⁰ is most especially a phenyl, also a naphthyl, such as 1- or 2-naphthyl, a biphenylyl, such as especially 4-biphenylyl, and also an anthryl, fluorenyl and azulenyl, as well as their aromatic analogues with one or more saturated rings. Preferred aryl-lower alkyl and -lower alkenyl radicals are, for example, phenyl-lower alkyl or phenyl-lower alkenyl with a terminal phenyl radical, such as for example benzyl, phenethyl, 1-, 2-, or 3-phenylpropyl, diphenylmethyl(benzhydryl), trityl, and cinnamyl, and also 1- or 2-naphthylmethyl. Aryl may be unsubstituted or substituted.

Heterocyclic radicals, including heterocyclic-acyclic radicals, are especially monocyclic, but also bi- or polycyclic, aza-, thia-, oxa-, thiaza-, oxaza-, diaza-, triaza-, or tetrazacyclic radicals of an aromatic character, as well as corresponding heterocyclic radicals of this type which are partly or most especially wholly saturated; if need be, for example as in the case of the above-mentioned carbocyclic or aryl radicals, these radicals may carry further acyclic, carbocyclic, or heterocyclic radicals and/or may be mono-, di-, or polysubstituted by functional groups. The acyclic part in heterocyclic-acyclic radicals has for example the meaning indicated for the corresponding carbocyclic-acyclic radicals. Most especially they are unsubstituted or substituted monocyclic radicals with a nitrogen, oxygen, or sulfur atom, such as 2-aziridinyl, and especially aromatic radicals of this type, such as pyrrolyl, for example 2-pyrrolyl or 3-pyrrolyl, pyridyl, for example 2-, 3-, or 4-pyridyl, and also thienyl, for example 2- or 3-thienyl, or furyl, for example 2-furyl; analogous bicyclic radicals with an oxygen, sulfur, or nitrogen atom are, for example, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, or benzo-thienyl, typically 2- or 3-benzothienyl; preferred monocyclic and bicyclic radicals with several heteroatoms are, for example, imidazolyl, typically 2-imidazolyl, pyrimidinyl, typically 2-or 4-pyrimidinyl, oxazolyl, typically 2-oxazolyl, isoxazolyl, typically 3-isoxazolyl, or thiazolyl, typically 2-thiazolyl, and benzimidazolyl, typically 2-benzimidazolyl, benzoxazolyl, typically 2-benzoxazolyl, or quinazolyl, typically 2-quinazolinyl. Appropriate partially or, especially, completely saturated analogous radicals may also be considered, such as 2-tetrahydrofuryl, 4-tetrahydrofuryl, 2- or 3-pyrrolidyl, 2-, 3-, or 4-piperidyl, and also 2-or 3-morpholinyl, 2- or 3-thiomorpholinyl, 2-piperazinyl, and N,N′-bis-lower alkyl-2-piperazinyl radicals. These radicals may also carry one or more acyclic, carbocyclic, or heterocyclic radicals, especially those mentioned hereinabove. Heterocyclic-acyclic radicals are especially derived from acyclic radicals with a maximum of 7, preferably a maximum of 4 carbon atoms, for example those named hereinabove, and may carry one, two, or more heterocyclic radicals, for example those named hereinabove, the ring possibly being linked to the aliphatic chain also by one of its nitrogen atoms.

As already mentioned, a hydrocarbyl (including a heterocyclyl) may be substituted by one, two, or more identical or different substituents (functional groups); one or more of the following substituents may be considered: lower alkyl; free, etherified and esterified hydroxyl groups; carboxy groups and esterified carboxy groups; mercapto- and lower alkylthio- and, if need be, substituted phenylthio groups; halogen atoms, typically chlorine and fluorine, but also bromine and iodine; halogen-lower alkyl groups; oxo groups which are present in the form of formyl (i.e. aldehydo) and keto groups, also as corresponding acetals or ketals; azido groups; nitro groups; cyano groups; primary, secondary and preferably tertiary amino groups, amino-lower alkyl, mono- or disubstituted amino-lower alkyl, primary or secondary amino groups protected by conventional protecting groups (especially lower alkoxycarbonyl, typically tert-butoxycarbonyl) lower alkylenedioxy, and also free or functionally modified sulfo groups, typically sulfamoyl or sulfo groups present in free form or as salts. The hydrocarbyl radical may also carry carbamoyl, ureido, or guanidino groups, which are free or which carry one or two substituents, and cyano groups. The above use of the word “groups” is taken to imply also an individual group.

Halogen-lower alkyl contains preferably 1 to 3 halogen atoms; preferred is trifluoromethyl or chloromethyl.

An etherified hydroxyl group present in the hydrocarbyl as substituent is, for example, a lower alkoxy group, typically the methoxy-, ethoxy-, propoxy-, isopropoxy-, butoxy-, and tert-butoxy group, which may also be substituted, especially by (i) heterocyclyl, whereby heterocyclyl can have preferably 4 to 12 ring atoms, may be unsaturated, or partially or wholly saturated, is mono- or bicyclic, and may contain up to three heteroatoms selected from nitrogen, oxygen, and sulfur, and is most especially pyrrolyl, for example 2-pyrrolyl or 3-pyrrolyl, pyridyl, for example 2-, 3- or 4-pyridyl, and also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2-or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2-or 3-morpholinyl, 2- or 3-thiomorpholinyl, 2-piperazinyl or N,N′-bis-lower alkyl-2-piperazinyl; and also (ii) by halogen atoms, for example mono-, di-, or polysubstituted especially in the 2-position, as in the 2,2,2-trichloroethoxy, 2-chloroethoxy, or 2-iodoethoxy radical, or (iii) by hydroxy or (iv) lower alkoxy radicals, each preferably monosubstituted, especially in the 2-position, as in the 2-methoxyethoxy radical. Such etherified hydroxyl groups are also unsubstituted or substituted phenoxy radicals and phenyl-lower alkoxy radicals, such as especially benzyloxy, benzhydryloxy, and triphenylmethoxy(trityloxy), as well as heterocyclyloxy radicals, wherein heterocyclyl can have preferably 4 to 12 ring atoms, may be unsaturated, or partially or wholly saturated, is mono- or bicyclic, and may contain up to three heteroatoms selected from nitrogen, oxygen, and sulfur, and is most especially pyrrolyl, for example 2-pyrrolyl or 3-pyrrolyl, pyridyl, for example 2-, 3- or 4-pyridyl, and also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2- or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2-or 3-morpholinyl, 2- or 3-thiomorpholinyl, 2-piperazinyl or N,N′-bis-lower alkyl-2-piperazinyl; such as especially 2- or 4-tetrahydropyranyloxy.

Etherified hydroxyl groups in this context are taken to include silylated hydroxyl-groups, typically for example tri-lower alkylsilyloxy, typically trimethylsilyloxy and dimethyl-tert-butylsilyloxy, or phenyldi-lower alkylsilyloxy and lower alkyl-diphenylsilyloxy.

An esterified hydroxyl group present in the hydrocarbyl as a substituent is, for example, lower alkanoyloxy.

A carboxyl group present in the hydrocarbyl as a substituent is one in which the hydrogen atom is replaced by one of the hydrocarbyl radicals characterised hereinabove, preferably a lower alkyl- or phenyl-lower alkyl radical; an example of an esterified carboxyl group is lower alkoxycarbonyl or phenyl-lower alkoxycarbonyl substituted if need be in the phenyl part, especially the methoxy, ethoxy, tert-butoxy, and benzyloxycarbonyl group, as well as a lactonised carboxyl group.

A primary amino group —NH₂ as substituent of the hydrocarbyls may also be present in a form protected by a conventional protecting group. A secondary amino group carries, instead of one of the two hydrogen atoms, a hydrocarbyl radical, preferably an unsubstituted one, typically one of the above-named, especially lower alkyl, and may also be present in protected form.

A tertiary amino group present in the hydrocarbyl as substituent carries 2 different or, preferably, identical hydrocarbyl radicals (including the heterocyclic radicals), such as the unsubstituted hydrocarbyl radicals characterised hereinabove, especially lower alkyl.

A preferred amino group is one with the formula R₁₁(R₁₂)N—, wherein R₁₁ and R₁₂ are independently in each case hydrogen, unsubstituted acyclic C₁-C₇hydrocarbyl (such as especially C₁-C₄alkyl or C₂-C₄alkenyl) or monocyclic aryl, aralkyl, or aralkenyl, substituted if necessary by C₁-C₄-alkyl, C₁-C₄-alkoxy, halogen, and/or nitro, and having a maximum of 10 carbon atoms, where the carbon-containing radicals may be interlinked through a carbon-carbon bond or an oxygen atom, a sulfur atom, or a nitrogen atom substituted if necessary by hydrocarbyl. In such a case, they form a nitrogen-containing heterocyclic ring with the nitrogen atom of the amino group. The following are examples of especially preferred disubstituted amino groups: di-lower alkylamino, typically dimethylamino or diethylamino, pyrrolidino, imidazol-1-yl, piperidino, piperazino, 4-lower alkylpiperazino, morpholino, thiomorpholino and piperazino or 4-methylpiperazino, as well as diphenylamino and dibenzylamino substituted if need be, especially in the phenyl part, for example by lower-alkyl, lower-alkoxy, halogen, and/or nitro; of the protected groups, especially lower alkoxycarbonylamino, typically tert-butoxycarbonylamino, phenyl-lower alkoxycarbonylamino, typically 4-methoxybenzyloxycarbonylamino, and 9-fluorenylmethoxycarbonylamino.

Amino-lower alkyl is most especially substituted in the 1-position of the lower alkyl chain by amino and is especially aminomethyl.

Mono- or disubstituted amino-lower alkyl is amino-lower alkyl substituted by one or two radicals, wherein amino-lower alkyl is most especially substituted by amino in the 1-position of the lower alkyl chain and is especially aminomethyl; the amino substituents here are preferably (if 2 substituents are present in the respective amino group independently of one another) from the group comprising lower alkyl, such as especially methyl, ethyl or n-propyl, hydroxy-lower alkyl, typically 2-hydroxyethyl, C₃-C₈cycloalkyl, especially cyclohexyl, amino-lower alkyl, typically 3-aminopropyl or 4-aminobutyl, N-mono- or N,N-di(lower alkyl)-amino-lower alkyl, typically 3-(N,N-dimethylamino)propyl, amino, N-mono- or N,N-di-lower alkylamino and N-mono- or N,N-di-(hydroxy-lower alkyl)amino.

Disubstituted amino-lower alkyl is also a 5 or 6-membered, saturated or unsaturated heterocyclyl bonded to lower alkyl via a nitrogen atom (preferably in the 1-position) and having 0 to 2, especially 0 or 1, other heteroatoms selected from oxygen, nitrogen, and sulfur, which is unsubstituted or substituted, especially by one or two radicals from the group comprising lower alkyl, typically methyl, and also oxo. Preferred here is pyrrolidino (1-pyrrolidinyl), piperidino (1-pi-peridinyl), piperazino(1-piperazinyl), 4-lower alkylpiperazino, typically 4-methylpiperazino, imidazolino(1-imidazolyl), morpholino(4-morpholinyl), or also thiomorpholino, S-oxo-thio-morpholino, or S,S-dioxothiomorpholino.

Lower alkylenedioxy is especially methylenedioxy.

A carbamoyl group carrying one or two substituents is especially aminocarbonyl(carbamoyl) which is substitiuted by one or two radicals at the nitrogen; the amino substituents here are preferably (if 2 substituents are present in the respective amino group independently of one another) from the group comprising lower alkyl, such as especially methyl, ethyl or n-propyl, hydroxy-lower alkyl, typically 2-hydroxyethyl, C₃-C₈cycloalkyl, especially cyclohexyl, amino-lower alkyl, typically 3-aminopropyl or 4-aminobutyl, N-mono- or N,N-di(lower alkyl)-amino-lower alkyl, typically 3-(N,N-dimethylamino)propyl, amino, N-mono- or N,N-di-lower alkylamino and N-mono- or N,N-di-(hydroxy-lower alkyl)amino; disubstituted amino in aminocarbamoyl is also a 5 or 6-membered, saturated or unsaturated heterocyclyl with a bonding nitrogen atom and 0 to 2, especially 0 or 1, other heteroatoms selected from oxygen, nitrogen, and sulfur, which is unsubstituted or substituted, especially by one or two radicals from the group comprising lower alkyl, typically methyl, and also oxo. Preferred here is pyrrolidino(1-pyrrolidinyl), piperidino(1-piperidinyl), piperazino(1-piperazinyl), 4-lower alkylpiperazino, typically 4-methylpiperazino, imidazolino(1-imidazolyl), morpholino(4-morpholinyl), or also thiomorpholino, S-oxo-thiomorpholino, or S,S-dioxothiomorpholino.

An acyl derived from an organic sulfonic acid, which is designated Ac², is especially one with the subformula R^{o}—SO₂—, wherein R^{o} is a hydrocarbyl as defined above in the general and specific meanings, the latter also being generally preferred here. Especially preferred is lower alkylphenylsulfonyl, especially 4-toluenesulfonyl.

An acyl derived from a phosphoric acid, esterified if necessary, which is designated Ac³, is especially one with the subformula R^{o}O(R^{o}O)P(═O)—, wherein the radicals R^{o} are, independently of one another, as defined in the general and specific meanings indicated above.

Reduced data on substituents given hereinbefore and hereinafter are considered to be preferences.

Preferred compounds according to the invention are, for example, those wherein R^{o} has the following preferred meanings: lower alkyl, especially methyl or ethyl, amino-lower alkyl, wherein the amino group is unprotected or is protected by a conventional amino protecting group—especially by lower alkoxycarbonyl, typically tert-lower alkoxycarbonyl, for example tert-butoxycarbonyl—e.g. aminomethyl, R,S-, R- or preferably S-1-aminoethyl, tert-butoxycarbonylaminomethyl or R,S-, R-, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, cyano-lower alkyl, typically 2-cyanoethyl, tetrahydropyranyloxy-lower alkyl, typically 4-(tetrahydropyranyl)-oxymethyl, morpholino-lower alkyl, typically 2-(morpholino)ethyl, phenyl, lower alkylphenyl, typically 4-methylphenyl, lower alkoxyphenyl, typically 4-methoxyphenyl, imidazolyl-lower alkoxyphenyl, typically 4-[2-(imidazol-1-yl)ethyl)oyxphenyl, carboxyphenyl, typically 4-carboxyphenyl, lower alkoxycarbonylphenyl, typically 4-ethoxycarbonylphenyl or 4-methoxyphenyl, halogen-lower alkylphenyl, typically 4-chloromethylphenyl, pyrrolidinophenyl, typically 4-pyrrolidinophenyl, imidazol-1-ylphenyl, typically 4-(imidazolyl-1-yl)phenyl, piperazinophenyl, typically 4-piperazinophenyl, (4-lower alkylpiperazino)phenyl, typically 4-(4-methylpiperazino)phenyl, morpholinophenyl, typically 4-morpholinophenyl, pyrrolidino-lower alkylphenyl, typically 4-pyrrolidinomethylphenyl, imidazol-1-yl-lower alkylphenyl, typically 4-(imidazolyl-1-ylmethyl)phenyl, piperazino-lower alkylphenyl, typically 4-piperazinomethylphenyl, (4-lower alkylpiperazinomethyl)-phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkyl-piperazino)phenyl, typically 4-(4-methylpiperazino)phenyl.

Preferred acyl radicals Ac¹ are acyl radicals of a carboxylic acid which are characterised by the subformula R^{o}—CO—, wherein R^{o} has one of the above general and preferred meanings of the hydrocarbyl radical R^{o}. Especially preferred radicals R^{o} here are lower alkyl, especially methyl or ethyl, amino-lower alkyl, wherein the amino group is unprotected or protected by a conventional amino protecting group, especially by lower alkoxycarbonyl, typically tert-lower alkoxycarbonyl, for example tert-butoxycarbonyl, e.g. aminomethyl, R,S-, R-, or preferably S-1-aminoethyl, tert-butoxycarbonylaminomethyl or R,S-, R-, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, tetrahydropyranyloxy-lower alkyl, typically 4-(tetrahydropyranyl)oxymethyl, phenyl, imidazolyl-lower alkoxyphenyl, typically 4-[2-(imidazol-1-yl)ethyl]oyxphenyl, carboxyphenyl, typically 4-carboxyphenyl, lower alkoxycarbonylphenyl, typically 4-ethoxycarbonylphenyl, halogen-lower alkylphenyl, typically 4-chloromethylphenyl, imidazol-1-ylphenyl, typically 4-(imidazolyl-1-yl)phenyl, pyrrolidino-lower alkylphenyl, typically 4-pyrrolidinomethylphenyl, piperazino-lower alkylphenyl, typically 4-piperazinomethylphenyl, (4-lower alkylpiperazinomethyl)phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkylpiperazino)phenyl, typically 4-(4-methylpiperazino)phenyl.

A further preferred Acyl Ac¹ is derived from monoesters of carbonic acid and is characterised by the subformula R^{o}—O—CO—. The lower alkyl radicals, especially tent-butyl, are especially preferred hydrocarbyl radicals R^{o} in these derivatives.

Another preferred Acyl Ac¹ is derived from amides of carbonic acid (or also thiocarbonic acid) and is characterised by the formula R^{o}HN—C(═W)— or R^{o}R^{o}N—C(═W)—, wherein the radicals R^{o} are, independently of one another, as defined above and W is sulfur and especially oxygen. In particular, compounds are preferred wherein Ac¹ is a radical of formula R^{o}HN—C(═W)—, wherein W is oxygen and R^{o} has one of the following preferred meanings: morpholino-lower alkyl, typically 2-morpholinoethyl, phenyl, lower alkoxyphenyl, typically 4-methoxyphenyl or 4-ethoxy-phenyl, carboxyphenyl, typically 4-carboxyphenyl, or lower alkoxycarbonylphenyl, typically 4-ethoxycarbonylphenyl.

A preferred acyl Ac² of subformula R^{o}—SO₂—, wherein R^{o} is a hydrocarbyl as defined in the above general and specific meanings, is lower alkylphenylsulfonyl, typically 4-toluenesulfonyl.

If p is 0, the nitrogen atom bonding R₃ is uncharged. If p is 1, then R₄ must also be present, and the nitrogen atom bonding R₃ and R₄ (quaternary nitrogen) is then positively charged.

The definitions for an aliphatic, carbocyclic, or carbocyclic-aliphatic radical with up to 29 carbon atoms each, or for a heterocyclic or heterocyclic-aliphatic radical with up to 20 carbon atoms each and up to 9 heteroatoms each, or acyl with up to 30 carbon atoms each, preferably match the definitions given for the corresponding radicals R₃ and R₄. Especially preferred is R₅ lower-alkyl, especially methyl, or most especially hydrogen.

Z is especially lower alkyl, most especially methyl or hydrogen.

If the two bonds indicated by wavy lines are missing in ring A, then no double bonds (tetra-hydrogenated derivatives) are present between the carbon atoms characterised in formula I by the numbers 1, 2, 3, and 4, but only single bonds, whereas ring B is aromatic (double bonds between the carbon atoms characterised in formula I by 8 and 9 and those characterised by 10 and 11). If the two bonds indicated by wavy lines are missing in ring B, then no double bonds (tetra-hydrogenated derivatives) are present between the carbon atoms characterised in formula I by the numbers 8, 9, 10, and 11, but only single bonds, whereas ring A is aromatic (double bonds between the carbon atoms characterised in formula I by 1 and 2 and those characterised by 3 and 4). If the total of four bonds indicated by wavy lines are missing in rings A and B, and are replaced by a total of 8 hydrogen atoms, then no double bonds (octa-hydrogenated derivatives) are present between the carbon atoms numbered 1, 2, 3, 4, 8, 9, 10, and 11 in formula I, but only single bonds.

By their nature, the compounds of the invention may also be present in the form of pharmaceutically, i.e. physiologically, acceptable salts, provided they contain salt-forming groups. For isolation and purification, pharmaceutically unacceptable salts may also be used. For therapeutic use, only pharmaceutically acceptable salts are used, and these salts are preferred.

Thus, compounds of formula I having free acid groups, for example a free sulfo, phosphoryl or carboxyl group, may exist as a salt, preferably as a physiologically acceptable salt with a salt-forming basic component. These may be primarily metal or ammonium salts, such as alkali metal or alkaline earth metal salts, for example sodium, potassium, magnesium or calcium salts, or ammonium salts with ammonia or suitable organic amines, especially tertiary monoamines and heterocyclic bases, for example triethylamine, tri-(2-hydroxyethyl)-amine, N-ethylpiperidine or N,N′-dimethylpiperazine.

Compounds of the invention having a basic character may also exist as addition salts, especially as acid addition salts with inorganic and organic acids, but also as quaternary salts. Thus, for example, compounds which have a basic group, such as an amino group, as a substituent may form acid addition salts with common acids. Suitable acids are, for example, hydrohalic acids, e.g. hydrochloric and hydrobromic acid, sulfuric acid, phosphoric acid, nitric acid or perchloric acid, or aliphatic, alicyclic, aromatic or heterocyclic carboxylic or sulfonic acids, such as formic, acetic, propionic, succinic, glycolic, lactic, malic, tartaric, citric, fumaric, maleic, hydroxymaleic, oxalic, pyruvic, phenylacetic, benzoic, p-aminobenzoic, anthranilic, p-hydroxybenzoic, salicylic, p-aminosalicylic acid, pamoic acid, methanesulfonic, ethanesulfonic, hydroxyethanesulfonic, ethylenedisulfonic, halobenzenesulfonic, toluenesulfonic, naphthalenesulfonic acids or sulfanilic acid, and also methionine, tryptophan, lysine or arginine, as well as ascorbic acid.

In view of the close relationship between the compounds (especially of formula I) in free form and in the form of their salts, including those salts that can be used as intermediates, for example in the purification or identification of the novel compounds, and of their solvates, any reference hereinbefore and hereinafter to the free compounds is to be understood as referring also to the corresponding salts, and the solvates thereof, e.g,. hydrates, as appropriate and expedient.

The compounds of formula A, B, C, D, I, II, III, IV, V or VI especially those wherein R₅ is hydrogen, possess valuable pharmacological properties.

In the case of the groups of radicals or compounds mentioned hereinbefore and hereinafter, general definitions may, insofar as appropriate and expedient, be replaced by the more specific definitions stated hereinbefore and hereinafter.

Preference is given to a compounds of formula I, II, III, IV, V, VI wherein:

R₁ and R₂ independently of each other are lower alkyl, lower alkyl substituted by halogen, C₆-C₁₄aryl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkylcarbamoyl, sulfo, lower alkanesulfonyl, lower alkoxysulfonyl, aminosulfonyl, N-lower alkylaminosulfonyl or N,N-di-lower alkylaminosulfonyl; halogen; lower alkoxy; C₆-C₁₄aryloxy; C₆-C₁₄aryl-lower alkoxy; lower alkanoyloxy; C₆-C₁₄arylcarbonyloxy; amino monosubstituted or disubstituted by lower alkyl, C₆-C₁₄aryl, C₆-C₁₄aryl-lower alkyl, lower alkanoyl or C₆-C₁₂arylcarbonyl; cyano; nitro; mercapto; lower alkylthio; C₆-C₁₄arylthio; C₆-C₁₄aryl-lower alkylthio; lower alkanoylthio; C₆-C₁₄aryl-lower alkanoylthio; carboxy; lower alkoxycarbonyl, C₆-C₁₄aryl-lower alkoxycarbonyl; C₆-C₁₄aryloxycarbonyl; carbamoyl; carbamoyl N-mono- or N,N-disubstituted by lower alkyl, C₆-C₁₄aryl or C₆-C₁₄aryl-lower alkyl; sulfo; C₆-C₁₄arylsulfonyl; C₆-C₁₄aryl-lower alkanesulfonyl; lower alkanesulfonyl; or aminosulfonyl N-mono- or N,N-disubstituted by lower alkyl, C₆-C₁₄aryl or C₆-C₁₄aryl-lower alkyl, wherein C₆-C₁₄aryl is an aryl radical with 6 to 12 carbon atoms in the ring system, which may be unsubstituted or substituted by halogen, phenyl or naphthyl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkylcarbamoyl, sulfo, lower alkanesulfonyl, lower alkoxysulfonyl, aminosulfonyl, N-lower alkylaminosulfonyl or N,N-di-lower alkylaminosulfonyl;

n and m are independently of each other 0 or 1 or 2, preferably 0;

R₃, R₄, R₈, R₁₀ are independently of each other hydrogen, lower alkyl, lower alkenyl or lower alkadienyl, which are each unsubstituted or monosubstituted or polysubsituted, preferably monosubstituted or disubstituted by a substituent independently selected from lower alkyl; hydroxy; lower alkoxy, which may be unsubstituted or mono-, di-, or trisubstituted by (i) heterocyclyl with 4 to 12 ring atoms, which may be unsaturated, wholly saturated, or partly saturated, is monocyclic or bicyclic and may contain up to three heteroatoms selected from nitrogen, oxygen and sulfur, and is most especially pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzo-thienyl, typically 2- or 3-benzothienyl; imidazolyl, typically 1- or 2-imidazolyl, pyrimidinyl, typically 2-or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 4-tetrahydropyranyl, 1-, 2- or 3-pyrrolidyl, 1-, 2-, 3-, or 4-piperidyl, 1-, 2-or 3-morpholinyl, 2- or 3-thiomorpholinyl, 2-piperazinyl or N,N′-bis-lower alkyl-2-piperazinyl, (ii) by halogen, (iii) by hydroxy or (iv) by lower alkoxy; phenoxy; phenyl-lower alkoxy; heterocyclyloxy, wherein heterocyclyl is pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzo-thienyl, typically 2- or 3-benzothienyl; imidazolyl, typically 1- or 2-imidazolyl, pyrimidinyl, typically 2-or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2-or 3-morpholinyl, 2- or 3-thiomorpholinyl, 2-piperazinyl or N,N′-bis-lower alkyl-2-piperazinyl, such as especially 2- or 4-tetrahydropyranyloxy; lower alkanoyloxy; carboxy; lower alkoxycarbonyl; phenyl-lower alkoxycarbonyl; mercapto; lower alkylthio; phenylthio; halogen; halogen-lower alkyl; oxo (except in the 1-position, because otherwise acyl); azido; nitro; cyano; amino; mono-lower alkylamino; di-lower alkylamino; pyrrolidino; imidazol-1-yl; piperidino; piperazino; 4-lower alkylpiperazino; morpholino; thiomorpholino; diphenylamino or dibenzylamino unsubstituted or substituted in the phenyl part by lower alkyl, lower alkoxy, halogen and/or nitro; lower alkoxycarbonylamino; phenyl-lower alkoxycarbonylamino unsubstituted or substituted in the phenyl part by lower alkyl or lower alkoxy; fluorenylmethoxycarbonylamino; amino-lower alkyl; monosubstituted or disubstituted amino-lower alkyl, wherein the amino substituent is selected from lower alkyl, hydroxy-lower alkyl, C₃-C₈cycloalkyl, amino-lower alkyl, N-mono- or N,N-di(-lower alkyl)amino-lower alkyl, amino, N-mono- or N,N-di-lower alkylamino and N-mono- or N,N-di-(hydroxy-lower alkyl)amino; pyrrolidino-lower alkyl; piperidino-lower alkyl; piperazino-lower alkyl; 4-lower alkylpiperazino-lower alkyl; imidazol-1-yl-lower alkyl; morpholino-lower alkyl; thiomorpholino-lower alkyl; S-oxo-thiomorpholino-lower alkyl; S,S-dioxothiomorpholino-lower alkyl; lower alkylendioxy; sulfamoyl; sulfo; carbamoyl; ureido; guanidino; cyano; aminocarbonyl(carbamoyl) and aminocarbonyloxy, which are substituted by one or two radicals on the nitrogen, wherein the amino substituents are selected independently of one another from the group comprising lower alkyl, hydroxy-lower alkyl, C₃-C₈cycloalkyl, amino-lower alkyl, N-mono- or N,N-di(-lower alkyl)amino-lower alkyl, amino, N-mono- or N,N-di-lower alkylamino and N-mono- or N,N-di-(hydroxy-lower alkyl)amino; pyrrolidinocarbonyl; piperidinocarbonyl; piperazinocarbonyl; 4-lower alkylpiperazinocarbonyl; imidazolinocarbonyl; morpholinocarbonyl; thiomorpholinocarbonyl; S-oxo-thiomorpholinocarbonyl; and S,S-dioxothiomorpholino;

phenyl, naphthyl, phenyl-lower alkyl or phenyl-lower alkenyl with a terminal phenyl radical, which is unsubstituted or monosubstituted or disubstituted by the radicals named above as substituents of lower alkyl, lower alkenyl or lower alkadienyl;

or heterocyclyl-lower alkyl, wherein heterocyclyl is pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2-or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2-or 3-morpholinyl, 2- or 3-thiomorpholinyl, 2-piperazinyl or N,N′-bis-lower alkyl-2-piperazinyl, which in each case are unsubstituted or monosubstituted or disubstituted by the radicals named above as substituents of lower alkyl, lower alkenyl, or lower alkadienyl;

or acyl of the subformula Y—C(═W)—, wherein W is oxygen and Y is hydrogen, R^{o}, R^{o}—O—, R^{o}HN—, or R^{o} R^{o} N— (wherein the radicals R^{o} may be the same or different),

or

acyl of the subformula R^{o}—SO₂—,

whereby R₄ may also be absent for the compound of formula II;

or

R₄ is absent for compounds of formula II, hydrogen or CH₃ for compounds of formula I, and

R₃ is acyl of the subformula Y—C(═W)—, wherein W is oxygen and Y is hydrogen, R^{o}, R^{o}—O—, R^{o}HN—, or R^{o}R^{o}N— (wherein the radicals R^{o} may be the same or different),

or

is acyl of the subformula R^{o}—SO₂—,

wherein R⁰ in the said radicals has the following meanings: substituted or unsubstituted lower alkyl, especially methyl or ethyl, amino-lower alkyl hydroxy-lower alkyl, wherein the amino group is unprotected or is protected by a conventional amino protecting group—especially by lower alkoxycarbonyl, typically tert-lower alkoxycarbonyl, for example tert-butoxycarbonyl—e.g. aminomethyl, R,S-, R- or preferably S-1-aminoethyl, tert-butoxycarbonylaminomethyl or R,S-, R-, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, cyano-lower alkyl, typically 2-cyanoethyl, tetrahydropyranyloxy-lower alkyl, typically 4-(tetrahydropyranyl)oxymethyl, morpholino-lower alkyl, typically 2-(morpholino)ethyl, phenyl, lower alkylphenyl, typically 4-methylphenyl, lower alkoxyphenyl, typically 4-methoxyphenyl, imidazolyl-lower alkoxyphenyl, typically 4-[2-(imidazol-1-yl)ethyl)oxyphenyl, carboxyphenyl, typically 4-carboxyphenyl, lower alkoxycarbonylphenyl, typically 4-ethoxycarbonylphenyl or 4-methoxyphenyl, halogen-lower alkylphenyl, typically 4-chloromethylphenyl, pyrrolidinophenyl, typically 4-pyrrolidinophenyl, imidazol-1-ylphenyl, typically 4-(imidazolyl-1-yl)phenyl, piperazinophenyl, typically 4-piperazinophenyl, (4-lower alkylpiperazino)phenyl, typically 4-(4-methylpiperazino)phenyl, morpholinophenyl, typically 4-morpholinophenyl, pyrrolidino-lower alkylphenyl, typically 4-pyrrolidinomethylphenyl, imidazol-1-yl-lower alkylphenyl, typically 4-(imidazolyl-1-ylmethyl)phenyl, piperazino-lower alkylphenyl, typically 4-piperazinomethylphenyl, (4-lower alkylpiperazinomethyl)-phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkylpiperazino)phenyl, typically 4-(4-methylpiperazino)phenyl.

p is 0 if R₄ is absent, or is 1 if R₃ and R₄ are both present and in each case are one of the aforementioned radicals (for compounds of formula II);

R₅ is hydrogen or lower alkyl, especially hydrogen,

X stands for 2 hydrogen atoms, for O, or for 1 hydrogen atom and hydroxy; or for 1 hydrogen atom and lower alkoxy;

Z is hydrogen or especially lower alkyl, most especially methyl;

and for compounds for formula II, either the two bonds characterised by wavy lines are preferably absent in ring A and replaced by 4 hydrogen atoms, and the two wavy lines in ring B each, together with the respective parallel bond, signify a double bond;

or also the two bonds characterised by wavy lines are absent in ring B and replaced by a total of 4 hydrogen atoms, and the two wavy lines in ring A each, together with the respective parallel bond, signify a double bond;

or both in ring A and in ring B all of the 4 wavy bonds are absent and are replaced by a total of 8 hydrogen atoms;

or a salt thereof, if at least one salt-forming group is present.

Particular preference is given to a compound of formula I wherein;

m and n are each 0;

R₃ and R₄ are independently of each other hydrogen, lower alkyl unsubstituted or mono- or disubstituted, especially monosubstituted, by radicals selected independently of one another from carboxy; lower alkoxycarbonyl; and cyano;

or

R₄ is hydrogen or —CH₃, and

R₃ is as defined above or preferably R₃ is, acyl of the subformula R^{o}—CO, wherein R^{o} is lower alkyl; amino-lower alkyl, wherein the amino group is present in unprotected form or is protected by lower alkoxycarbonyl; tetrahydropyranyloxy-lower alkyl; phenyl; imidazolyl-lower alkoxyphenyl; carboxyphenyl; lower alkoxycarbonylphenyl; halogen-lower alkylphenyl; imidazol-1-ylphenyl; pyrrolidino-lower alkylphenyl; piperazino-lower alkylphenyl; (4-lower alkylpiperazinomethyl)phenyl; morpholino-lower alkylphenyl; piperazinocarbonylphenyl; or (4-lower alkylpiperazino)phenyl;

or is acyl of the subformula R^{o}—O—CO—, wherein R^{o} is lower alkyl;

or is acyl of the subformula R^{o}HN—C(═W)—, wherein W is oxygen and R^{o} has the following meanings: morpholino-lower alkyl, phenyl, lower alkoxyphenyl, carboxyphenyl, or lower alkoxycarbonylphenyl;

or R₃ is lower alkylphenylsulfonyl, typically 4-toluenesulfonyl;

further specific examples of preferred R₃ groups are described below for the preferred compounds of formula II,

R₅ is hydrogen or lower alkyl, especially hydrogen,

X stands for 2 hydrogen atoms or for O;

Z is methyl or hydrogen;

or a salt thereof, if at least one salt-forming group is present.

Particular preference is given to a compound of formula II wherein

m and n are each 0;

R₃ and R₄ are independently of each other hydrogen, lower alkyl unsubstituted or mono- or disubstituted, especially monosubstituted, by radicals selected independently of one another from carboxy; lower alkoxycarbonyl; and cyano; whereby R₄ may also be absent;

or

R₄ is absent, and

R₃ is acyl from the subformula R^{o}—CO, wherein R^{o} is lower alkyl, especially methyl or ethyl; amino-lower alkyl, wherein the amino group is unprotected or protected by lower alkoxy-carbonyl, typically tert-lower alkoxycarbonyl, for example tert-butoxycarbonyl, e.g. aminomethyl, R,S-, R-, or preferably S-1-aminoethyl, tert-butoxycarbonylaminomethyl or R,S-, R-, or preferably S-1-(tert-butoxycarbonylamino)ethyl; tetrahydropyranyloxy-lower alkyl, typically 4-(tetrahydropyranyl)oxymethyl; phenyl; imidazolyl-lower alkoxyphenyl, typically 4-[2-(imidazol-1-yl)ethyl)oyxphenyl; carboxyphenyl, typically 4-carboxyphenyl; lower alkoxycarbonylphenyl, typically 4-methoxy- or 4-ethoxycarbonylphenyl; halogen-lower alkylphenyl, typically 4-chloromethylphenyl; imidazol-1-ylphenyl, typically 4-(imidazolyl-1-yl)phenyl; pyrrolidino-lower-alkylphenyl, typically 4-pyrrolidinomethylphenyl; piperazino-lower alkylphenyl, typically 4-piperazinomethylphenyl; (4-lower alkylpiperazinomethyl)phenyl, typically 4-(4-methylpiperazinomethyl)phenyl; morpholino-lower alkylphenyl, typically 4-morpholinomethylphenyl; piperazinocarbonylphenyl, typically 4-piperazinocarbonylphenyl; or (4-lower alkylpiperazino)phenyl, typically 4-(4-methylpiperazino)phenyl;

or is acyl of the subformula R^{o}—O—CO—, wherein R^{o} is lower alkyl;

or is acyl of the subformula R^{o}HN—C(═W)—, wherein W is oxygen and R^{o} has the following preferred meanings: morpholino-lower alkyl, typically 2-morpholinoethyl, phenyl, lower alkoxyphenyl, typically 4-methoxyphenyl or 4-ethoxyphenyl, carboxyphenyl, typically 4-carboxyphenyl, or lower alkoxycarbonylphenyl, typically 4-ethoxycarbonylphenyl;

or is lower alkylphenylsulfonyl, typically 4-toluenesulfonyl;

p is 0 if R₄ is absent, or is 1 if R₃ and R₄ are both present and in each case are one of the aforementioned radicals;

R₅ is hydrogen or lower alkyl, especially hydrogen,

X stands for 2 hydrogen atoms or for O;

Z is methyl or hydrogen;

and either the two bonds characterised by wavy lines are preferably absent in ring A and replaced by 4 hydrogen atoms, and the two wavy lines in ring B each, together with the respective parallel bond, signify a double bond;

or also the two bonds characterised by wavy lines are absent in ring B and replaced by a total of 4 hydrogen atoms, and the two wavy lines in ring A each, together with the respective parallel bond, signify a double bond;

or both in ring A and in ring B all of the 4 wavy bonds are absent and are replaced by a total of 8 hydrogen atoms;

or a salt thereof, if at least one salt-forming group is present.

Most especially preferred compounds of formula II are selected from;
8,9,10,11-Tetrahydrostaurosporine;
N-[4-(4-methylpiperaziN-1-ylmethypbenzoyl]-1,2,3,4-tetrahydrostaurosporine;
N-(4-chloromethylbenzoyl)-1,2,3,4-tetrahydrostaurosporine;
N-(4-(pyrrolidin-1-ylmethypbenzoyl)-1,2,3,4-tetrahydrostaurosporine;
N-(4-(morpholin-4-ylmethyl)benzoyl)-1,2,3,4-tetrahydrostaurosporine;
N-(4-(piperazin-1-ylmethyl)benzoyl)-1,2,3,4-tetrahydrostaurosporine;
N-ethyl-1,2,3,4-tetrahydrostaurosporine;
N-tosyl-1,2,3,4-tetrahydrostaurosporine;
N-triflouroacetyl-1,2,3,4-tetrahydrostaurosporine;
N-[4-(2-imidazol-1-yl-ethoxy)benzoyl]-1,2,3,4-tetrahydrostaurosporine;
N-methoxycarbonylmethyl-1,2,3,4-tetrahydrostaurosporine;
N-carboxymethyl-1,2,3,4-tetrahydrostaurosporine;
N-terephthaloylmethyl ester-1,2,3,4-tetrahydrostaurosporine;
N-terephthaloyl-1,2,3,4-tetrahydrostaurosporine;
N-(4-ethylpiperazinylcarbonylbenzoyl)-1,2,3,4-tetrahydrostaurosporine;
N-(2-cyanoethyl)-1,2,3,4-tetrahydrostaurosporine;
N-benzoyl-1,2,3,4-tetrahydrostaurosporine;
N,N-dimethyl-1,2,3,4-tetrahydrostaurosporinium iodide;
N-BOC-glycyl-1,2,3,4-tetrahydrostaurosporine;
N-glycyl-1,2,3,4-tetrahydrostaurosporine;
N-(3-(tert-butoxycarbonyl)propyl)-1,2,3,4-tetrahydrostaurosporine;
N-(3-carboxypropyl)-1,2,3,4-tetrahydrostaurosporine;
N-(4-imidazol-1-yl)benzoyl]-1,2,3,4-tetrahydrostaurosporine;
N-[(tetrahydro-2h-pyran-4-yloxy)acetyl]-1,2,3,4-tetrahydrostaurosporine;
N-BOC-1-alanyl-1,2,3,4-tetrahydrostaurosporine;
N-1-alanyl-1,2,3,4-tetrahydrostaurosporine hydrochloride;
N-methyl-1,2,3,4-tetrahydro-6-methylstaurosporine;
N-(4-carboxyphenylaminocarbonyl)-1,2,3,4-tetrahydrostaurosporine;
N-(4-ethylphenylaminocarbonyl)-1,2,3,4-tetrahydrostaurosporine;
N-(N-phenylaminocarbonyl)-1,2,3,4-tetrahydrostaurosporine;
N-(N-[2-(1-morpholino)ethyl]aminocarbonyl)-1,2,3,4-tetrahydrostaurosporine;
N-(N-[4-methoxyphenyl]aminocarbonyl)-1,2,3,4-tetrahydrostaurosporine;
1,2,3,4-tetrahydro-6-methylstaurosporine;
N-BOC-1,2,3,4-tetrahydrostaurosporine;
N-BOC-1,2,3,4-tetrahydro-6-methylstaurosporine;
N-BOC-1,2,3,4-tetrahydro-6-methyl-7-oxo-staurosporine;
1,2,3,4,8,9,10,11-octahydrostaurosporine;
   or a pharmaceutically acceptable salt thereof, if at least one salt-forming group is present.

Most especially preferred is the compound of formula I designated 1,2,3,4-tetrahydro-staurosporine, or a (particularly pharmaceutically acceptable) salt thereof (here, m and n in formula I are 0, R₃ is hydrogen, R₄ is absent, provided no salt is present (p=0), or is hydrogen if a salt is present (p=1), R₅ is hydrogen, the two bonds represented by wavy lines are absent in Ring A and are replaced by a total of 4 hydrogen atoms and the two bonds represented by wavy lines in Ring B are in each case a double bond together with the parallel bonds, X stands for 2 hydrogen atoms, and Z is methyl).

Most especially preferred are the compounds of formula A wherein;
A) X═O; R₁,R_{2,}R₅═H; Q=-(CH₂)₂—O—CH(CH₂)OH—(CH₂)₂—
B) X═O; R₁,R_{2,}R₅═H; Q=-(CH₂)₂—O—CH(CH₂N(CH₃)₂)—(CH₂)₂—
C) X=2 hydrogen atoms; R₁,R_{2,}R₅═H;

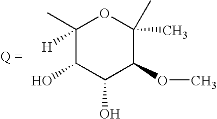

Most especially preferred are the compounds of formula I wherein;
A) X=b 2 hydrogen atoms; R₁,R_{2,}R₃,R₅═H; R₄═CH₃; Z═CH₃ (staurosporine)
B) X=1 hydrogen and 1 hydroxy atoms in (R) or (S) isomeric form; R₁,R_{2,}R₃,R₅═H; R₄═CH₃; Z═CH₃ (UCN-01 and UCN-02)
C) X=2 hydrogen atoms; R₁,R_{2,}R₅═H; R₄═CH₃; R_{3,}=benzoyl; Z═CH₃ (CGP41251 or PKC412 or MIDOSTAURIN)
D) X═O; R₁,R_{2,}R₅═H; R_{3,}═CH₃; R₄=ethyloxycarbonyl; Z═CH₃ (NA 382; CAS=143086-33-3)
E) X=1 hydrogen and 1 hydroxy atom; R₁,R_{2,}R₅═H; R₃═CH₃; Z═CH₃; and R₄ is selected from —(CH₂)₂OH; —CH₂CH(OH)CH₂OH; —CO(CH₂)₂CO₂Na; —(CH₂)₃CO₂H; —COCH₂N(CH₃)₂;

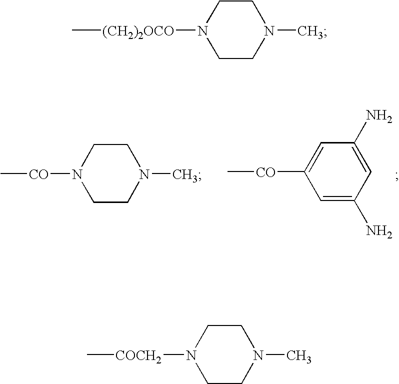

F) X=2 hydrogen atoms; R₁,R_{2,}R₅═H; R₃═CH₃; Z═CH₃; and R₄ is selected from N-[0-(tetrahydropyran-4-yl)-D-lactoyl]; N-[2-methyl-2-(tetrahydropyran-4-yloxy)-propionyl; N-[0-(tetrahydropyran-4-yl)-L-lactoyl]; N-[0-(tetrahydropyran-4-yl)-D-lactoyl]; N-[2-(tetrahydro-pyran-4-yloxy)-acetyl)]
G) X═O; R₁,R_{2,}R₅═H; R₃═CH₃; Z═CH₃; and R₄ is selected from N-[0-(tetrahydropyran-4-yl)-D-lactoyl]; N-[2-(tetrahydro-pyran-4-yloxy)-acetyl)]
H) X=1 hydrogen and 1 hydroxy atom; R₁,R_{2,}R₅═H; R₃═CH₃; Z═CH₃; and R₄ is selected from N-[0-(tetrahydropyran-4-yl)-D-lactoyl]; N-[2-(tetrahydro-pyran-4-yloxy)-acetyl)]

The abbreviation “CAS” means the CHEMICAL ABSTRACTS registry number.

The most preferred compounds of formula I e.g. MIDOSTAURIN [International Nonproprietary Name] are covered and have been specifically described by the European patent No. 0 296 110 published on Dec. 21, 1988, as well as in U.S. Pat. No. 5,093,330 published on Mar. 3, 1992, and Japanese Patent No. 2 708 047. Other preferred compounds are covered and described by the patent applications WO 95/32974 and WO 95/32976 both published on Dec. 7, 1995. All the compounds described in these documents are incorporated into the present application by reference.

Most especially preferred are the compounds of formula III wherein;
A) X=2 hydrogen atoms; R₁,R_{2,}R₅═H; R₆═CH₃; R₇=methyloxycarbonyl; Z═H (2-methyl K252a)
B) X=2 hydrogen atoms; R₁,R_{2,}R₅,R₆═H; R₇=methyloxycarbonyl; Z═H (K-252a)
C) X=2 hydrogen atoms; R₁,R_{2,}R₅,R₆═H; R₇=methyloxycarbonyl; Z═CH₃ (KT-5720)

Most especially preferred are the compounds of formula IV wherein;
A) X═O; R₁,R_{2,}R₅═H; R₉═CH₂—NMe₂; R₈═CH₃; m′=n′=2
B) X═O; R₁,R_{2,}R₅═H; R₉═CH₂—NH₂; R₈═CH₃; m′=2; n′=1 (Ro-31-8425; CAS=151342-35-7)

Most especially preferred are the compounds of formula V wherein;
A) X═O; R₁,R_{2,}R₅═H; R₈═CH₃; R₁₀=-(CH₂)₃—NH₂; (Ro-31-7549; CAS=138516-31)
B) X═O; R₁,R_{2,}R₅═H; R₈═CH₃; R₁₀=-(CH₂)₃—S—(C═NH)—NH₂; (Ro-31-8220; CAS=125314-64-9))
C) X═O; R₁,R_{2,}R₅═H; R₈═CH₃; R₁₀=-CH₃;

Most especially preferred are the compounds of formula VI wherein;
A) X=2 hydrogen atoms; R₁,R_{2,}R₅═H; R₄═CH₃; Z═CH₃; R₃ selected from methyl or (C₁-C₁₀)alkyl, arylmethyl, C₆H₂CH₂—

STAUROSPORINE DERIVATIVES and their manufacturing process have been specifically described in many prior documents, well known by the man skilled in the art.

Compounds of formula A, B, C, D and their manufacturing process have for instance, been described in the European patents No. 0 657 458 published on Jun. 14, 1995, in the European patents No. 0 624 586 published on Nov. 17, 1994, in the European patents No. 0 470 490 published on Feb. 12, 1992, in the European patents No. 0 328 026 published on Aug. 16, 1989, in the European patents No. 0 384 349 published on Aug. 29, 1990, as well as in many publications such as Barry M Trost* and Weiping Tang Org. Lett., 3(21), 3409-3411.

Compounds of formula I and their manufacturing processes have specifically been described in the European patents No. 0 296 110 published on Dec. 21, 1988, as well as in U.S. Pat. No. 5,093,330 published on Mar. 3, 1992, and Japanese Patent No. 2 708 047. Compounds of formula I having a tetrahydropyran-4-yl)-lactoyl substitution on R₄ have been described in the European patent No. 0 624 590 published on Nov. 17, 1994. Other compounds have been described in the European patent No. 0 575 955 published Dec. 29, 1993, European patent No. 0 238 011 published on Sep. 23, 1987 (UCN-O1), International patent application EP98/04141 published as WO99/02532 on Jul. 03, 1998.

Compounds of formula II and their manufacturing processes have specifically been described in the European patents No. 0 296 110 published on Dec. 21, 1988, as well as in U.S. Pat. No. 5,093,330 published on Mar. 3, 1992, and Japanese Patent No. 2 708 047.

Compounds of formula III and their manufacturing processes have specifically been described in the patent applications claiming the priority of the US patent application U.S. Pat. No. 920,102 filed on Jul. 24, 1992. (i.e European patents No. 0 768 312 published on Apr. 16, 1997, No. 1 002 534 published May 24, 2000, No. 0 651 754 published on May 10, 1995).

Compounds of formula IV and their manufacturing processes have specifically been described in the patent applications claiming the priority of the British patent applications GB 9309602 and GB 9403249 respectively filed on May 10, 1993, and on Feb. 21, 1994. (i.e European patents No. 0 624 586 published on Nov. 17, 1994, No. 1 002 534 published May 24, 2000, No. 0 651 754 published on May 10, 1995).

Compounds of formula V and their manufacturing processes have specifically been described in the patent applications claiming the priority of the British patent applications GB 8803048, GB 8827565, GB 8904161 and GB 8928210 respectively filed on Feb. 10, 1988, Nov. 25, 1988, Feb. 23, 1989 and Dec. 13, 1989. (i.e European patents No. 0 328 026 published on Aug. 16, 1989, and No. 0 384 349 published Aug. 29, 1990).

Compounds of formula VI and their manufacturing processes have specifically been described in the patent applications claiming the priority of the U.S. patent applications Ser. No. 07/777,395 (Con), filed on Oct. 10, 1991 (i.e International patent application WO 93/07153 published on Apr. 15, 1993).

In each case where citations of patent applications or scientific publications are given in particular for the STAUROSPORINE DERIVATIVE compounds, the subject-matter of the final products, the pharmaceutical preparations and the claims are hereby incorporated into the present application by reference to these publications.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The preferred STAUROSPORINE DERIVATIVE according to the invention is N-[(R9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide of the formula (VII):

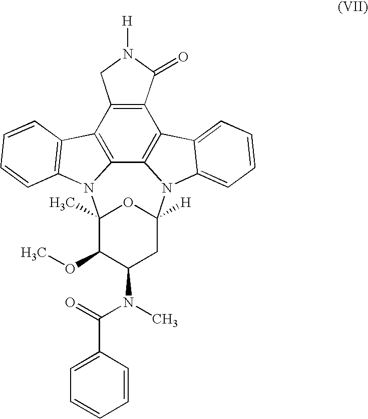

or a salt thereof, (hereinafter: “Compound of formula VII or MIDOSTAURIN”).

Compound of formula VII is also known as MIDOSTAURIN [International Nonproprietary Name] or PKC412.

MIDOSTAURIN is a derivative of the naturally occurring alkaloid staurosporine, and has been specifically described in the European patent No. 0 296 110 published on Dec. 21, 1988, as well as in U.S. Pat. No. 5,093,330 published on Mar. 3, 1992, and Japanese Patent No. 2 708 047.

Nucleic Acids

Nucleic acid molecules that inhibit apoptosis or programmed cell death include, but are not limited to, antisense oligonucleotides and RNAi constructs specific to the gene encoding the antiapoptotic MCL1 protein or to transcripts of this gene. Most preferred for use in the inventive combination is Mcl-1 specific RNAi construct, including especially a mcl-1 specific siRNA. Exemplary mcl-1 RNAi constructs of the invention are described in the examples.

The protein encoded by myeloid cell leukemia sequence 1 (BCL2-related) (“mcl-1”) belongs to the Bcl-2 family. Alternative splicing occurs at this gene locus, resulting in two transcript variants encoding distinct isoforms. The longer gene product (isoform 1) enhances cell survival by inhibiting apoptosis. The alternatively spliced shorter gene product (isoform 2) promotes apoptosis and is death-inducing. Aliases of the mcl-1 gene include but are not limited to EAT, MCL1L, MCL1S, MGC104264, MGC1839, and TM. Alternative designations of the MCL1 polypeptide include, but are not limited to: induced myeloid leukemia cell differentiation protein Mcl-1; myeloid cell leukemia sequence 1; and myeloid cell leukemia sequence 1, isoform 1.

As used herein, the term “nucleic acid molecule” is intended to include DNA molecules (e.g., cDNA or genomic DNA or oligonucleotides) and RNA molecules (e.g., mRNA or RNAi constructs such as siRNA, shRNA) and analogs of the DNA or RNA molecules generated using nucleoside analogs. The nucleic acid molecule can be single stranded or double stranded. Where single-stranded, the nucleic acid can be a sense strand or an antisense strand. The term includes synthetic (e.g., chemically synthesized) DNA. Nucleic acids can be synthesized using nucleoside analogs or derivatives (e.g., inosine or phosphorothioate nucleotides). Such nucleosides can be used, for example, to prepare nucleic acids that have altered base-pairing abilities or increased resistance to nucleases.

The invention encompasses RNAi or antisense nucleic acid molecules specific to a mcl1 nucleotide sequence, including, e.g., the mcl-1 cDNA. The cDNA sequence of the longer mcl-1 isoform 1 (GenBank accno. NM_{—}021960) is provided in SEQ ID NO:1.

| GenBank accno. NM_021960, 4020 bp linear mRNA, PRI 12-JUN.-2006 | |
|---|---|
| [Homo sapiens] | |
| (SEQ ID NO: 1) | |
| 1 | caccccgtag gactggccgc cctaaaaccg tgataaagga gctgctcgcc acttctcact |
| 61 | tccgcttcct tccagtaagg agtcggggtc ttccccagtt ttctcagcca ggcggcggcg |
| 121 | gcgactggca atgtttggcc tcaaaagaaa cgcggtaatc ggactcaacc tctactgtgg |
| 181 | gggggccggc ttgggggccg gcagcggcgg cgccacccgc ccgggagggc gacttttggc |
| 241 | tacggagaag gaggcctcgg cccggcgaga gataggggga ggggaggccg gcgcggtgat |
| 301 | tggcggaagc gccggcgcaa gccccccgtc caccctcacg ccagactccc ggagggtcgc |
| 361 | gcggccgccg cccattggcg ccgaggtccc cgacgtcacc gcgacccccg cgaggctgct |
| 421 | tttcttcgcg cccacccgcc gcgcggcgcc gcttgaggag atggaagccc cggccgctga |
| 481 | cgccatcatg tcgcccgaag aggagctgga cgggtacgag ccggagcctc tcgggaagcg |
| 541 | gccggctgtc ctgccgctgc tggagttggt cggggaatct ggtaataaca ccagtacgga |
| 601 | cgggtcacta ccctcgacgc cgccgccagc agaggaggag gaggacgagt tgtaccggca |
| 661 | gtcgctggag attatctctc ggtaccttcg ggagcaggcc accggcgcca aggacacaaa |
| 721 | gccaatgggc aggtctgggg ccaccagcag gaaggcgctg gagaccttac gacgggttgg |
| 781 | ggatggcgtg cagcgcaacc acgagacggc cttccaaggc atgcttcgga aactggacat |
| 841 | caaaaacgaa gacgatgtga aatcgttgtc tcgagtgatg atccatgttt tcagcgacgg |
| 901 | cgtaacaaac tggggcagga ttgtgactct catttctttt ggtgcctttg tggctaaaca |
| 961 | cttgaagacc ataaaccaag aaagctgcat cgaaccatta gcagaaagta tcacagacgt |
| 1021 | tctcgtaagg acaaaacggg actggctagt taaacaaaga ggctgggatg ggtttgtgga |
| 1081 | gttcttccat gtagaggacc tagaaggtgg catcaggaat gtgctgctgg cttttgcagg |
| 1141 | tgttgctgga gtaggagctg gtttggcata tctaataaga tagccttact gtaagtgcaa |
| 1201 | tagttgactt ttaaccaacc accaccacca ccaaaaccag tttatgcagt tggactccaa |
| 1261 | gctgtaactt cctagagttg caccctagca acctagccag aaaagcaagt ggcaagagga |
| 1321 | ttatggctaa caagaataaa tacatgggaa gagtgctccc cattgattga agagtcactg |
| 1381 | tctgaaagaa gcaaagttca gtttcagcaa caaacaaact ttgtttggga agctatggag |
| 1441 | gaggactttt agatttagtg aagatggtag ggtggaaaga cttaatttcc ttgttgagaa |
| 1501 | caggaaagtg gccagtagcc aggcaagtca tagaattgat tacccgccga attcattaat |
| 1561 | ttactgtagt gttaagagaa gcactaagaa tgccagtgac ctgtgtaaaa gttacaagta |
| 1621 | atagaactat gactgtaagc ctcagtactg tacaagggaa gcttttcctc tctctaatta |
| 1681 | gctttcccag tatacttctt agaaagtcca agtgttcagg acttttatac ctgttatact |
| 1741 | ttggcttggt ttccatgatt cttactttat tagcctagtt tatcaccaat aatacttgac |
| 1801 | ggaaggctca gtaattagtt atgaatatgg atatcctcaa ttcttaagac agcttgtaaa |
| 1861 | tgtatttgta aaaattgtat atatttttac agaaagtcta tttctttgaa acgaaggaag |
| 1921 | tatcgaattt acattagttt ttttcatacc cttttgaact ttgcaacttc cgtaattagg |
| 1981 | aacctgtttc ttacagcttt tctatgctaa actttgttct gttcagttct agagtgtata |
| 2041 | cagaacgaat tgatgtgtaa ctgtatgcag actggttgta gtggaacaaa tctgataact |
| 2101 | atgcaggttt aaattttctt atctgatttt ggtaagtatt ccttagatag gtttttcttt |
| 2161 | gaaaacctgg gattgagagg ttgatgaatg gaaattcttt cacttcatta tatgcaagtt |
| 2221 | ttcaataatt aggtctaagt ggagttttaa ggttactgat gacttacaaa taatgggctc |
| 2281 | tgattgggca atactcattt gagttccttc catttgacct aatttaactg gtgaaattta |
| 2341 | aagtgaattc atgggctcat ctttaaagct tttactaaaa gattttcagc tgaatggaac |
| 2401 | tcattagctg tgtgcatata aaaagatcac atcaggtgga tggagagaca tttgatccct |
| 2461 | tgtttgctta ataaattata aaatgatggc ttggaaaagc aggctagtct aaccatggtg |
| 2521 | ctattattag gcttgcttgt tacacacaca ggtctaagcc tagtatgtca ataaagcaaa |
| 2581 | tacttactgt tttgtttcta ttaatgattc ccaaaccttg ttgcaagttt ttgcattggc |
| 2641 | atctttggat ttcagtcttg atgtttgttc tatcagactt aaccttttat ttcctgtcct |
| 2701 | tccttgaaat tgctgattgt tctgctccct ctacagatat ttatatcaat tcctacagct |
| 2761 | ttcccctgcc atccctgaac tctttctagc ccttttagat tttggcactg tgaaacccct |
| 2821 | gctggaaacc tgagtgaccc tccctcccca ccaagagtcc acagaccttt catctttcac |
| 2881 | gaacttgatc ctgttagcag gtggtaatac catgggtgct gtgacactaa cagtcattga |
| 2941 | gaggtgggag gaagtccctt ttccttggac tggtatcttt tcaactattg ttttatcctg |
| 3001 | tctttggggg caatgtgtca aaagtcccct caggaatttt cagaggaaag aacattttat |
| 3061 | gaggctttct ctaaagtttc ctttgtatag gagtatgctc acttaaattt acagaaagag |
| 3121 | gtgagctgtg ttaaacctca gagtttaaaa gctactgata aactgaagaa agtgtctata |
| 3181 | ttggaactag ggtcatttga aagcttcagt ctcggaacat gacctttagt ctgtggactc |
| 3241 | catttaaaaa taggtatgaa taagatgact aagaatgtaa tggggaagaa ctgccctgcc |
| 3301 | tgcccatctc agagccataa ggtcatcttt gctagagcta tttttaccta tgtatttatc |
| 3361 | gttcttgatc ataagccgct tatttatatc atgtatctct aaggacctaa aagcacttta |
| 3421 | tgtagttttt aattaatctt aagatctggt tacggtaact aaaaaagcct gtctgccaaa |
| 3481 | tccagtggaa acaagtgcat agatgtgaat tggtttttag gggccccact tcccaattca |
| 3541 | ttaggtatga ctgtggaaat acagacaagg atcttagttg atattttggg cttggggcag |
| 3601 | tgagggctta ggacacccca agtggtttgg gaaaggagga ggggagtggt gggtttatag |
| 3661 | ggggaggagg aggcaggtgg tctaagtgct gactggctac gtagttcggg caaatcctcc |
| 3721 | aaaagggaaa gggaggattt gcttagaagg atggcgctcc cagtgactac tttttgactt |
| 3781 | ctgtttgtct tacgcttctc tcagggaaaa acatgcagtc ctctagtgtt tcatgtacat |
| 3841 | tctgtggggg gtgaacacct tggttctggt taaacagctg tacttttgat agctgtgcca |
| 3901 | ggaagggtta ggaccaacta caaattaatg ttggttgtca aatgtagtgt gtttccctaa |
| 3961 | ctttctgttt ttcctgagaa aaaaaaataa atcttttatt caaaaaaaaa aaaaaaaaaa |

The invention further encompasses nucleic acid molecules that differ from the nucleotide sequence of SEQ ID NO:1 due to degeneracy of the genetic code but encode the same mcl-1 protein as that encoded by SEQ ID NO:1. The 350 aa MCL1 polypeptide (GenPep accno. NP_{—}068779) encoded by the nucleotide of SEQ ID NO:1 is provided as SEQ ID NO:2.

| GenPept accno. NP_068779 350 aa myeloid cell leukemia sequence 1 | |
|---|---|
| isoform 1 PRI 12-JUN.-2006 [Homo sapiens]. | |
| (SEQ ID NO: 2) | |
| 1 | mfglkrnavi glnlycggag lgagsggatr pggrllatek easarreigg geagaviggs |
| 61 | agasppstlt pdsrrvarpp pigaevpdvt atparllffa ptrraaplee meapaadaim |
| 121 | speeeldgye peplgkrpav lpllelvges gnntstdgsl pstpppaeee edelyrqsle |
| 181 | iisrylreqa tgakdtkpmg rsgatsrkal etlrrvgdgv qrnhetafqg mlrkldikne |
| 241 | ddvkslsrvm ihvfsdgvtn wgrivtlisf gafvakhlkt inqesciepl aesitdvlvr |
| 301 | tkrdwlvkqr gwdgfveffh vedleggirn vllafagvag vgaglaylir |

It will be appreciated by skilled practitioners that DNA sequence polymorphisms that lead to changes in the amino acid sequences of mcl-1 may exist within a given population (e.g., a human population). Such genetic polymorphisms may exist within a population due to natural allelic variation. Such natural allelic variations can typically result in 15% variance in the nucleotide sequence of the mcl-1 gene. Any and all such nucleotide variations, whether “silent” or resulting in amino acid polymorphisms that are the result of natural allelic variation and that do not alter the functional activity of mcl-1, are intended to be within the scope of the invention. Thus, e.g., 1%, 2%, 3%, 4%, or 5% of the amino acids in mcl-1 can be replaced by another amino acid, e.g., by conservative substitutions.

In addition to naturally occurring allelic variants of the mcl-1 sequence that may exist in the population, skilled practitioners will appreciate that changes can be introduced by mutating the nucleotide sequence of SEQ ID NO:1, thereby leading to changes in the amino acid sequence of the encoded protein without altering the functional ability of the protein. For example, one can make nucleotide substitutions leading to amino acid substitutions at “non essential” amino acid residues. A “non essential” amino acid residue is a residue that can be altered from the wild type sequence of human mcl-1 protein without altering the biological activity of the mcl-1 protein, whereas an “essential” amino acid residue is required for biological activity. For example, amino acid residues that are conserved among mcl-1 proteins of various species are predicted to be particularly unamenable to alteration.

Accordingly, nucleic acid molecules encoding mcl-1 proteins that contain changes in amino acid residues that are not essential for activity are included in the present invention. Such mcl-1 proteins differ in amino acid sequence from SEQ ID NO:2, and yet retain at least a portion of the wild-type's biological activity. In one embodiment, for example, the isolated nucleic acid molecule includes a sequence encoding a protein that includes an amino acid sequence at least about 75% identical, e.g., 80%, 85%, 90%, 95% or 98% identical to the amino acid sequence of SEQ ID NO:2, over the entire length of SEQ ID NO:2.

An isolated nucleic acid molecule encoding a mcl-1 protein having a sequence differing from SEQ ID NO:2 can be created by introducing one or more nucleotide substitutions, additions or deletions into SEQ ID NO:1, such that one or more amino acid substitutions, additions or deletions are introduced into the encoded protein. Mutations can be introduced by standard techniques, e.g., site directed mutagenesis and PCR mediated mutagenesis. Conservative amino acid substitutions can be made at one or more predicted non essential amino acid residues. Thus, for example, up to 1%, 2%, 3%, 5%, or 10% of the amino acids can be replaced by conservative substitution. A “conservative amino acid substitution” is one in which an amino acid residue is replaced with another having a similar side chain. Families of amino acid residues having similar side chains are known in the art. They include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta branched side chains (e.g., threonine, valine, isoleucine) and aromatic side chains (e.g., tyrosine, phenylalanine, tryptophan, histidine). Thus, a predicted nonessential amino acid residue in mcl-1 can be replaced with another amino acid residue from the same side chain family. Alternatively, mutations can be introduced randomly along all or part of a mcl-1 coding sequence, such as by saturation mutagenesis, and the resulting mutants can be screened for mcl-1 biological activity to identify mutants that retain activity. Following mutagenesis, the protein can be expressed and its activity determined.

The present invention also encompasses antisense nucleic acids molecules, i.e., molecules that are complementary to a sense strand, e.g., complementary to the coding strand of a double stranded cDNA molecule or complementary to an mRNA sequence. An antisense nucleic acid can hydrogen bond to the corresponding sense strand. The antisense nucleic acid can be complementary to the entire mcl-1-coding sequence, or to only a portion thereof. An antisense nucleic acid molecule can be antisense to a noncoding region of the sense strand. The noncoding regions (“5′ and 3′ untranslated regions”) are the 5′ and 3′ sequences that flank the coding region of a gene or mRNA and are not translated. The antisense nucleic acid molecule can be complementary to the entire coding region of mcl-1 mRNA, or, in some instances, complementary to only a portion of the coding or 3′ or 5′ noncoding region of mcl-1 mRNA. An antisense oligonucleotide can be, for example, about 5, e.g., about 10, 15, 18, 20, 25, 30, 35, 40, 45 or about 50 nucleotides in length. For general guidance concerning antisense nucleic acids, see, e.g., Goodchild, “Inhibition of Gene Expression by Oligonucleotides,” in Topics in Molecular and Structural Biology, Vol. 12: Oligodeoxynucleotides (Cohen, ed.), MacMillan Press, London, pp. 53-77 (1989). Exemplary useful antisense oligonucleotides include, but are not limited to, antisense oligonucleotides that include a sequence of at least 12 nucleotides complementary to a region between nucleotides 131 and 1183 of SEQ ID NO:1. See also SEQ ID NOS:7-9 in the Examples.

An antisense nucleic acid of the invention can be constructed using chemical synthesis, enzymatic ligation reactions and other art-known procedures. For example, an antisense nucleic acid (e.g., an antisense oligonucleotide) can be synthesized using naturally occurring nucleotides or variously modified nucleotides designed to increase the biological stability of the molecules and/or physical stability of the duplex formed between antisense and sense nucleic acids. For example, phosphorothioate derivatives and acridine substituted nucleotides can be used. Exemplary modified nucleotides useful for generating antisense nucleic acids include 5 fluorouracil, 5 bromouracil, 5 chlorouracil, 5 iodouracil, hypoxanthine, xanthine, 4 acetylcytosine, 5 (carboxyhydroxylmethyl)uracil, 1 methylguanine, 5 carboxymethylaminomethyl 2 thiouridine, 2,2 dimethylguanine, dihydrouracil, beta D galactosylqueosine, 5 carboxymethylaminomethyluracil, inosine, N6 isopentenyladenine, 1 methylinosine, 2 methyladenine, 2 methylguanine, 4 thiouracil, 3 methylcytosine, 5 methylcytosine, N6 adenine, 7 methylguanine, 5 methoxyuracil, 5 methylaminomethyluracil, 5 methoxyaminomethyl 2 thiouracil, pseudouracil, queosine, beta D mannosylqueosine, 5′ methoxycarboxymethyluracil, 5 methyl 2 thiouracil, 2 methylthio N6 isopentenyladenine, uracil 5 oxyacetic acid (v), wybutoxosine, 2 thiocytosine, 2 thiouracil, 5 methyluracil, uracil 5 oxyacetic acid methylester, 5 methyl 2 thiouracil, 3 (3 amino 3 N 2 carboxypropyl)uracil, (acp3)w, and 2,6 diaminopurine. Alternatively, an antisense nucleic acid can be produced using an expression vector, into which a nucleic acid has been subcloned in an antisense orientation (i.e., RNA transcribed from the inserted nucleic acid will be complementary to a target nucleic acid of interest, described further in the following subsection).

The antisense nucleic acid molecules of the invention can be administered to a subject or generated in situ such that they hybridize with or bind to cellular mRNA and/or genomic DNA encoding a mcl-1 protein. In this way, the antisense nucleic acids can inhibit expression of the protein, e.g., by inhibiting transcription and/or translation. Hybridization can be by conventional nucleotide complementarity to form a stable duplex or, in the case of an antisense nucleic acid molecule that binds to DNA duplexes, through specific interactions in the major groove of the double helix.

Antisense nucleic acids can be administered by any method known in the art, e.g., by direct injection at a tissue site. Alternatively or in addition, antisense nucleic acid molecules can be adapted to target select cells, so that they can be administered systemically. For systemic administration, antisense molecules can be modified such that they bind specifically to receptors or antigens expressed on a selected cell surface, e.g., by linking the antisense nucleic acid molecules to peptides or antibodies that bind to cell surface receptors or antigens. Antisense nucleic acid molecules can also be delivered to cells using certain vectors described herein. Vector constructs in which the sequence to be transcribed is placed under the control of a strong pol II or pol III promoter can be used to achieve sufficient intracellular concentrations of antisense transcripts.

An antisense nucleic acid molecule of the invention can be an a anomeric nucleic acid molecule. An α anomeric nucleic acid molecule forms specific double stranded hybrids with complementary RNA in which, contrary to the usual β units, the strands run parallel to each other (Gaultier et al. (1987) Nucleic Acids Res 15:6625 6641). The antisense nucleic acid molecule can also include a 2′o methylribonucleotide (Inoue et al. (1987) Nucleic Acids Res 15:6131 6148) or a chimeric RNA DNA analogue (Inoue et al. (1987) FEBS Lett 215:327 330).

The invention also includes nucleic acids capable of suppressing expression of mcl-1 by way of RNA interference (RNAi). RNAi is a process whereby double-stranded RNA (dsRNA) induces the sequence-specific degradation of homologous mRNA in animals and plant cells (Hutvagner and Zamore (2002) Cur. Opin Gene. Dev 12:225-232; Sharp (2001) Genes Dev. 15:485-490. In mammalian cells, RNAi can be triggered by, e.g., 21-nucleotide (nt) duplex RNAs, also called small inhibitory RNAs (siRNAs) (Chiu et al (2001) Mol Cell 10:549-561; Elbashir et al. (2001) Nature 411:494-498; or by micro-RNA (miRNA), functional small-hairpin RNA (shRNA), or dsRNAs that are expressed in vivo using DNA templates with RNA polymerase III promoters (Zeng et al. (2002) Mol Cell 9:1327-1333; Paddison et al. (2002) Genes Dev 16:948-958; Lee et al. (2002) Nature Biotechnol 20:500-505; Paul et al. (2002) Nature Biotechnol 20:505-508; Tuschl (2002) Nature Biotechnol 20:440-448; Yu et al. (2002) Proc Natl Acad Sci USA 99:6047-6052; McManus et al. (2002) RNA 8:842-850; Sui et al. (2002) Proc Natl Acad Sci USA 99:5515-5520; see also Hannon (2002) Nature 418:244-251.

The suppression, also known as “silencing,” is predominantly a cytoplasmic, post-transcriptional event that is evolutionarily conserved (see Hutvagner and Zamore (2002) Curr Opin Genet Dev 12:225-232). Current models suggest that dsRNA is processed into siRNA by the RNAse III enzyme, Dicer. The siRNA then forms a complex called RNA-induced silencing complex (RISC). This complex interacts with the target mRNA, which is then cleaved and degraded (Martinez et al. (2002) Cell 110:563-574; Schwarz et al. (2002) Mol Cell 10:537-548). shRNAs have been studied in mammalian cells (Paul et al. (2002) Genes & Dev 16:948-958), and can be expressed effectively in human cells (Sui et al. (2002) Nature Biotechnol 20:505-508). A DNA vector-based RNAi technology to suppress gene expression in mammalian cells is described by Yu and Turner ((2002) Proc Natl Acad Sci USA 99:5515-5520). RNAi by expression of siRNAs and shRNAs in mammalian cells has been described by Yu et al. ((2002) Proc Natl Acad Sci USA 99:6047-6052).

The present invention includes siRNAs, duplexes thereof (i.e., dsRNAs), shRNAs, and miRNAs, which interact with (e.g., bind) a mcl-1 target sequence (e.g., a mcl-1 mRNA (e.g., an mRNA corresponding to human mcl-1 sequences such as those shown herein)). Such nucleic acids can be, e.g., chemically synthesized RNA oligonucleotides (see Elbashir et al., supra). Exemplary RNAi sequences encompassed by and useful in the present invention include at least one of the following sequences, or its complementary strand sequence:

(mcl1, SEQ ID NO: 7)
5′ TTG GCT TTG TGT CCT TGG CG 3′
(mcl1, SEQ ID NO: 8)
5′ AAG AAA CGC GGU AAU CGG ACU 3′;
(mcl1, SEQ ID NO: 9)
5′ AGU CCG AUU ACC GCG UUU CUU 3′;
(luciferase, SEQ ID NO: 10)
5′ CUU ACG CUG AGU ACU UCG A 3′;

Plasmids can be used to transcribe shRNAs that can function as siRNAs (see Sui et al. (2002) Proc Natl Acad Sci USA 99:5515-5520; Brummelkamp et al. (2002) Science 296:550-553). Such plasmids can contain, e.g., an RNA polymerase III promoter, followed by sequence that is transcribed into an RNA containing a sense sequence, a loop structure, and an antisense sequence.

A variety of loops have been successfully employed, as have several polymerase III promoters (Brummelkamp et al., Science 296:550-553, 2002; Miyagishi and Taira, Nucl. Acids Res. 2:113-114, 2002). Vectors (e.g., plasmids) containing a promoter (e.g., an RNA polymerase III promoter) operably linked to a sequence that is transcribed into an RNA containing a mcl-1 “sense” sequence, a loop structure, and the corresponding mcl-1 “antisense” sequence can be made using procedures that are routine in the art. As U6-promoter-driven siRNAs with four uridine 3′ overhangs have been shown to suppress expression of a target gene in mammalian cells (Paddison et al. (2002) Nature Biotechnol 20:497-500), vectors of the present invention can include a U6 promoter and sequences that are transcribed into siRNAs with uridine overhangs (e.g., 1-6 uridines at the 3′ terminus). An exemplary shRNA encompassed by and useful in the present invention is described in the Examples section, which is transcribed from the sequences set forth herein as SEQ ID NOs: 9 and 10, respectively (see Table 1, below).

Typically, miRNAs are excised from an approximately 70-nucleotide precursor RNA stem-loop (the shRNA), possibly by the RNase III-type enzyme known as Dicer or a homolog thereof. By substituting the stem sequences of the miRNA precursor with miRNA sequence complementary to the target mRNA (here, a mcl-1 mRNA), a vector construct that expresses the miRNA can be used to produce siRNAs to initiate RNAi against the target in mammalian cells (Zeng, supra).

The nucleic acids of the invention can be included in, and expressed by, viral vectors. Accordingly, the invention features viral vectors that induce specific silencing of mcl-1 through expression of siRNA. For example, mcl-1-specific adenoviral vectors can be constructed by generating recombinant adenoviruses expressing siRNA under RNA Pol II promoter transcription control.

A siRNAs or other nucleic acid-based inhibitor can be administered to animals and humans using any art-known method. For example, a “high-pressure” delivery technique can be used, such as a rapid injection (for example, an injection that is complete within a few seconds) of a large volume of an siRNA-containing solution into the animal via an artery or vein (see, e.g., Lewis (2002) Nature Genetics 32:107-108). Microparticles, nanoparticles and liposomes can also be used to deliver siRNAs. siRNAs that target mcl-1 mRNA and that are associated with a microparticle, nanoparticle or liposome, or that are otherwise formulated for delivery to a biological cell, are within the scope of the present invention.

A dsRNA molecule of the present invention can include about 15 or more (e.g., 12, 13, 14, 15, 16, 17, 18, 19, 20, 21, 22, 23, 24, 25, 26, 27, 28, 29, 30 or more) nucleotides in each strand. The strands of the dsRNA can be substantially complementary to a target region of mcl-1 mRNA.

For example, one strand can be about 90% (e.g., about 90%, 95%, or 100%) complementary to a target region of mcl-1 mRNA. The target region can be any region of the mcl-1 nucleic acid sequence, e.g., a region corresponding to nucleotides 1 to 228 of SEQ ID NO:1, e.g., within the region encoding a polypeptide of amino acids 1 to 76 of SEQ ID NO:2.

A dsRNA molecule of the invention can be produced by, e.g., chemically synthesizing the molecule, transcribing the molecule in vitro from a DNA template, making the molecule in vivo from, e.g., shRNA (generation of dsRNAs and other inhibitory nucleic acids is discussed further above), or using any other method known in the art. For example, each strand of a dsRNA can be chemically synthesized using naturally occurring nucleotides or variously modified nucleosides designed to increase the biological stability of the molecules or to increase the physical stability of the duplex formed between the antisense and sense strands of the dsRNA (e.g., phosphorothioate derivatives and acridine substituted nucleosides can be used). The dsRNA can also be produced using an expression vector into which a nucleic acid including both the sense and antisense sequences in the same strand has been cloned (e.g., an RNA transcribed from the inserted nucleic acid may form a hairpin, the stem of which forms the dsRNA that includes an antisense strand that is complementary to a mcl-1 RNA). Accordingly, one can make a dsRNA that inhibits mcl-1 expression by generating (or designing) a dsRNA that includes a sequence that is the reverse and complement of a given mcl-1 mRNA sequence.

The dsRNA can be tested in mcl-1-expressing cells. An anti-mcl-1 dsRNA will decrease expression of mcl-1 (i.e., will decrease the levels of mcl-1 protein and mcl-1 mRNA) in the cells. mcl-1 expression can be assayed by methods known in the art. For example, levels of mcl-1 protein can be assayed by Western blot analysis, by immunoprecipitation, by ELISA, or by a functional assay. Levels of mcl-1 mRNA can be assayed by Northern blot, in situ analysis, or reverse transcription coupled with polymerase chain reaction (RT-PCR).

The invention also encompasses ribozymes. Ribozymes are catalytic RNA molecules with ribonuclease activity that are capable of cleaving a single stranded nucleic acid (e.g., mRNA) to which they have a complementary region. Thus, ribozymes (e.g., hammerhead ribozymes (described in Haselhoff and Gerlach (1988) Nature 334:585 591)) can be used to catalytically cleave mcl-1 mRNA transcripts, to thereby inhibit translation of mcl-1 mRNA. A ribozyme having specificity for a mcl-1 encoding nucleic acid can be designed based upon the nucleotide sequence of a mcl-1 cDNA disclosed herein. For example, a derivative of a Tetrahymena L 19 IVS RNA can be constructed in which the nucleotide sequence of the active site is complementary to the nucleotide sequence to be cleaved in a mcl-1 encoding mRNA. See, e.g., Cech et al. U.S. Pat. No. 4,987,071; and Cech et al. U.S. Pat. No. 5,116,742. Alternatively, mcl-1 mRNA can be used to select a catalytic RNA having a specific ribonuclease activity from a pool of RNA molecules. See, e.g., Bartel and Szostak (1993) Science 261:1411 1418.

The invention also encompasses nucleic acid molecules that form triple helical structures. For example, mcl-1 gene expression can be inhibited by targeting nucleotide sequences complementary to a regulatory region of the mcl-1 gene (e.g., the mcl-1 promoter and/or enhancers) to form triple helical structures that prevent transcription of the mcl-1 gene in target cells. See generally, Helene (1991) Anticancer Drug Des. 6(6):569 84; Helene (1992) Ann. N.Y. Acad. Sci. 660:27 36; and Maher (1992) Bioassays 14(12):807 15.

In certain embodiments, the nucleic acid molecules of the invention are modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (see Hyrup et al. (1996) Bioorganic & Medicinal Chemistry 4(1):5 23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry O'Keefe et al. (1996) Proc. Natl. Acad. Sci. USA 93:14670 675.

In the instant invention, mcl-1 PNAs can be used for therapeutic and diagnostic applications. For example, PNAs can be used as antisense or antigene agents for sequence specific modulation of gene expression by, e.g., inducing transcription or translation arrest or inhibiting replication of the gene. mcl-1 PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996) supra); or as probes or primers for DNA sequence and hybridization (Hyrup (1996) supra; Perry O'Keefe et al. (1996) Proc. Natl. Acad. Sci. USA 93: 14670 675).

In another embodiment, mcl-1 PNAs can be modified, e.g., to enhance their stability or cellular uptake, by attaching lipophilic or other helper groups to PNA, by the formation of PNA DNA chimeras, or by the use of liposomes or other techniques of drug delivery known in the art. For example, mcl-1 PNA DNA chimeras can be generated that may combine the advantageous properties of PNA and DNA. Such chimeras allow DNA recognition enzymes, e.g., RNAse H and DNA polymerases, to interact with the DNA portion while the PNA portion provides high binding affinity and specificity. PNA DNA chimeras can be linked using linkers of appropriate lengths selected in terms of base stacking, number of bonds between the nucleobases, and orientation (Hyrup (1996) supra). The synthesis of PNA DNA chimeras can be performed as described in Hyrup (1996) supra and Finn et al. (1996) Nucleic Acids Research 24(17):3357 63. For example, a DNA chain can be synthesized on a solid support using standard phosphoramidite coupling chemistry and modified nucleoside analogs, e.g., 5′ (4 methoxytrityl)amino 5′ deoxy thymidine phosphoramidite, can be used as a linker between the PNA and the 5′ end of DNA (Mag et al. (1989) Nucleic Acid Res. 17:5973 88). PNA monomers are then coupled in a stepwise manner to produce a chimeric molecule with a 5′ PNA segment and a 3′ DNA segment (Finn et al. (1996) Nucleic Acids Research 24(17):3357 63). Alternatively, chimeric molecules can be synthesized with a 5′ DNA segment and a 3′ PNA segment (Peterser et al. (1975) Bioorganic Med. Chem. Lett. 5:1119 11124).

In other embodiments, the oligonucleotide may include other appended groups such as peptides (e.g., for targeting host cell receptors in vivo) or agents facilitating transport across the cell membrane (see, e.g., Letsinger et al. (1989) Proc. Natl. Acad. Sci. USA 86:6553 6556; Lemaitre et al. (1987) Proc. Natl. Acad. Sci. USA 84:648 652; PCT Publication No. WO 88/09810) or the blood brain barrier (see, e.g., PCT Publication No. WO 89/10134). In addition, oligonucleotides can be modified with hybridization triggered cleavage agents (see, e.g., Krol et al. (1988) Bio/Techniques 6:958 976) or intercalating agents (see, e.g., Zon (1988) Pharm. Res. 5:539 549). To this end, the oligonucleotide may be conjugated to another molecule, e.g., a peptide, hybridization triggered cross linking agent, transport agent or hybridization triggered cleavage agent.

Vectors, Host Cells and Transgenic Animals

Also included in the present invention are vectors, e.g., expression vectors, containing a mcl-1 nucleic acid (or a portion thereof). As used herein, the term “vector” refers to a nucleic acid molecule capable of transporting another nucleic acid to which it has been linked. One type of vector is a plasmid, i.e., a circular double stranded DNA loop into which additional DNA segments can be ligated. Another type is a viral vector, wherein additional DNA segments can be ligated into the viral genome. Certain vectors are capable of autonomous replication in a host cell into which they are introduced. Therefore, also included in the invention are host cells containing the mcl-1 nucleic acid containing vector. The choice of vector and host cell is routine to a skilled practicioner of the art.

The present invention provides a method of treating myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC), comprising administering to a mammalin need of such a treatment a therapeutically effective amount of a combination of a FLT-3 kinase inhibitor and mcl-1 inhibitor such as an antisense oligonucleotide or a mcl-1-specific RNAi construct, each in free form or in form of a pharmaceutically acceptable salt or prodrug, respectively.

Preferably the instant invention provides a method for treating mammals, especially humans, suffering from myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC) comprising administering to a mammal in need of such treatment an therapeutically effective amount of a combination of N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide of the formula (VII), or a pharmaceutically acceptable salt thereof and an antisense oligonucleotide or a mcl-1-specific RNAi construct.

In another embodiment, the instant invention relates to the use of a combination of a FLT-3 kinase inhibitor and antisense oligonucleotide or a mcl-1-specific RNAi construct for treating myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC).

In a further embodiment, the instant invention relates to the use of a combination of a FLT-3 kinase inhibitor and a antisense oligonucleotide or a mcl-1-specific RNAi construct for the preparation of a pharmaceutical composition for treating myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC).

According to the invention a combination of N-[9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide of the formula (VII), or a pharmaceutically acceptable salt thereof and an antisense oligonucleotide or a mcl-1-specific RNAi construct are the preferred combinations of a FLT-3 kinase inhibitor and an antisense oligonucleotide or a mcl-1-specific RNAi construct.

Abbreviations:

ASO Antisense oligonucleotide(s)

ASM aggressive systemic mastocytosis

BM bone marrow

cladribine 2-chlorodeoxyadenosine (=2CdA)

FCS fetal calf serum

ISM indolent systemic mastocytosis

MC mast cell(s)

MCL mast cell leukemia

Mcl-1 myeloid cell leukemia-1

MCS mast cell sarcoma

miRNA micro RNA

PB peripheral blood

PBS phosphate buffered saline

RNAi RNA interferance

RT-PCR reverse transcriptase polymerase chain reaction

shRNA short hairpin RNA

siRNA short interfering RNA

SM systemic mastocytosis

SSM systemic smouldering mastocytosis

TK tyrosine kinase

The combination of a FLT-3 kinase inhibitor and an antisense oligonucleotide or a mel-1-specific RNAi construct, each in free form or in form of a pharmaceutically acceptable salt or prodrug, respectively, for treating myelodysplastic syndromes, lymphomas and leukemias, in particular Systemic mastocytosis, and also acute myeloid leukemia (AML) and also solid tumors such as e.g. colorectal cancer (CRC) and non-small cell lung cancer (NSCLC) may be a free or fixed combination of the combination partners.

In one aspect, the present invention also relates to a combination, such as a combined preparation or a pharmaceutical composition, which comprises (a) a FLT-3 inhibitor, especially the FLT-3 inhibitors specifically mentioned hereinbefore, in particular those mentioned as being preferred, and (b) an an antisense oligonucleotide or a mel-1-specific RNAi construct, in which the active ingredients (a) and (b) are present in each case in free form or in the form of a pharmaceutically acceptable salt or suitable biopharmaceutical formulation, for simultaneous, concurrent, separate or sequential use. Suitable biopharmaceutical formulation are known to one skilled in the art, wherein the formulation isoptimized depending upon whether the therapeutical administration is of an RNAi construct or for an antisense oligonucleotide construct.

The term “a combined preparation” defines especially a “kit of parts” in the sense that the combination partners (a) and (b) as defined above can be dosed independently or by use of different fixed combinations with distinguished amounts of the combination partners (a) and (b), i.e., simultaneously, concurrently, separately or sequentially. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, severity of the disease, age, sex, body weight, etc. of the patients.

As mentioned above the precise dosage of the FLT-3 inhibitor and the antisense oligonucleotide or a mcl-1-specific RNAi construct be employed for treating the diseases and conditions mentioned hereinbefore depends upon several factors including the host, the nature and the severity of the condition being treated, the mode of administration. However, in general, satisfactory results are achieved when the FLT-3 inhibitor is administered parenterally, e.g., intraperitoneally, intravenously, intramuscularly, subcutaneously, intratumorally, or rectally, or enterally, e.g., orally, preferably intravenously or, preferably orally, intravenously at a daily dosage of 0.1 to 10 mg/kg body weight, preferably 1 to 5 mg/kg body weight. In human trials a total dose of 225 mg/day is most presumably the Maximum Tolerated Dose (MTD). A preferred intravenous daily dosage is 0.1 to 10 mg/kg body weight or, for most larger primates, a daily dosage of 200-300 mg. A typical intravenous dosage is 3 to 5 mg/kg, three to five times a week.

Most preferably, the FLT-3 inhibitors, especially MIDOSTAURIN, are administered orally, by dosage forms such as microemulsions, soft gels or solid dispersions in dosages up to about 250 mg/day, in particular 225 mg/day, administered once, twice or three times daily.

Usually, a small dose is administered initially and the dosage is gradually increased until the optimal dosage for the host under treatment is determined. The upper limit of dosage is that imposed by side effects and can be determined by trial for the host being treated.

The FLT-3 inhibitors the antisense oligonucleotide or a mcl-1-specific RNAi construct may be combined with one or more pharmaceutically acceptable carriers and, optionally, one or more other conventional pharmaceutical adjuvants and administered enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

The infusion solutions according to the present invention are preferably sterile. This may be readily accomplished, e.g. by filtration through sterile filtration membranes. Aseptic formation of any composition in liquid form, the aseptic filling of vials and/or combining a pharmaceutical composition of the present invention with a suitable diluent under aseptic conditions are well known to the skilled addressee.

The FLT-3 inhibitors and an antisense oligonucleotide or a mel-1-specific RNAi construct may be formulated into enteral and parenteral pharmaceutical compositions containing an amount of the active substance that is effective for treating the diseases and conditions named hereinbefore, such compositions in unit dosage form and such compositions comprising a pharmaceutically acceptable carrier.

Examples of useful compositions of FLT-3 inhibitors are described in the European patents No. 0 296 110, No. 0 657 164, No. 0 296 110, No.0 733 372, No.0 711 556, No.0 711 557.

The preferred compositions of FLT-3 inhibitors are described in the European patent No. 0 657 164 published on Jun. 14, 1995. The described pharmaceutical compositions comprise a solution or dispersion of compounds of formula I such as MIDOSTAURIN in a saturated polyalkylene glycol glyceride, in which the glycol glyceride is a mixture of glyceryl and polyethylene glycol esters of one or more C8-C18 saturated fatty acids.

Two manufacture processes of such compositions of FLT-3 inhibitors are described hereafter.

Composition A:

Gelucire 44/14 (82 parts) is melted by heating to 60° C. Powdered MIDOSTAURIN (18 parts) is added to the molten material. The resulting mixture is homogenised and the dispersion obtained is introduced into hard gelatin capsules of different size, so that some contain a 25 mg dosage and others a 75 mg dosage of the MIDOSTAURIN. The resulting capsules are suitable for oral administration.

Composition B:

Gelucire 44/14 (86 parts) is melted by heating to 60° C. Powdered MIDOSTAURIN (14 parts) is added to the molten material. The mixture is homogenised and the dispersion obtained is introduced into hard gelatin capsules of different size, so that some contain a 25 mg dosage and others a 75 mg dosage of the MIDOSTAURIN. The resulting capsules are suitable for oral administration.

Gelucire 44/14 available commercially from Gattefossé; is a mixture of esters of C8-C18 saturated fatty acids with glycerol and a polyethylene glycol having a molecular weight of about 1500, the specifications for the composition of the fatty acid component being, by weight, 4-10% caprylic acid, 3-9% capric acid, 40-50% lauric acid, 14-24% myristic acid, 4-14% palmitic acid and 5-15% stearic acid.

A preferred example of Gelucire formulation consists of:
Gelucire (44/14): 47 g
MIDOSTAURIN: 3.0 g filled into a 60 mL Twist off flask

A preferred example of soft gel will contain the following Microemulsion:

| | | |
|---|---|---|
| Cornoil glycerides | 85.0 | mg |
| Polyethylenglykol 400 | 128.25 | mg |
| Cremophor RH 40 | 213.75 | mg |
| MIDOSTAURIN | 25.0 | mg |
| DL alpha Tocopherol | 0.5 | mg |
| Ethanol absolute | 33.9 | mg |
| Total | 486.4 | mg |

However, it should be clearly understood that it is for purposes of illustration only.

It can be shown by the test methods described below that the combination of a FLT-3 inhibitor and an mcl-1 antisense oligonucleotide or a mel-1-specific RNAi construct are more effective than treatment with either of the agents alone. In these studies determined the cell cycle effects and apoptosis induced by a an antisense oligonucleotide or a mel-1-specific RNAi construct, and a FLT-3 kinase inhibitor, preferably 4-benzyl staurosporine, against Systemic mastocytosis (SM) are demostrated.

The expression and functional role of Mcl-1 in neoplastic human MC has is examined. It can be shown that primary neoplastic MC in all variants of SM including ASM and MCL as well as the MCL cell line HMC-1, express Mcl-1 in a constitutive manner. In addition, we show that targeting of Mcl-1 in these cells is associated with reduced growth and induction of apoptosis, and with an increased sensitivity to TK inhibitors including PKC412, 4-methyl-3-[[4-(3-pyridinyl)-2-pyrimidinyl]amino]-N-[5-(4-methyl-1H-imidazol-1-yl)-3-(trifluoromethyl)phenyl]benzamide, and imatinib.

A newly developed flow cytometry (FCM) assay, utilizing anti-FLT-3 or phospho (p)-FLT-3 antibody, is used to demonstrate that while MV4-11 (MV) cells express both FLT-3 and p-FLT-3, RS4-11 (RS) cells only express FLT-3 on their cell surface. Exposure to 20 to 200 nM of a preferred FLT-3 inhibitor induces cell cycle GI phase accumulation and, in a dose-dependent manner, significantly more apoptosis of MV than RS cells. This is associated with marked attenuation of p-FLT-3, p-AKT and p-ERK1/2 but not of FLT-3, AKT or ERK1/2 levels, as determined by Western analyses. The preferred FLT-3 inhibitor also inhibits the surface expression of p-FLT-3 but not of FLT-3 (as can be determined by FCM) on MV cells. In contrast to the preferred FLT-3 inhibitor, treatment with a preferred HDAI compound attenuates both FLT-3 and p-FLT-3 levels in a dose-dependent manner in MV and RS cells, as can be determined both by Western and FCM analyses. Exposure to a preferred HDAI compound (20 to 100 nM) also down regulates the levels of p-FLT-3, p-AKT and p-ERK1/2. Significantly, co-treatment with a preferred FLT-3 inhibitor and a preferred HDAI compound surprinsingly induces apoptosis of MV and RS cells. This is associated with more attenuation of p-FLT-3, p-AKT and p-ERK1/2 in MV cells.

Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a otherwise non-effective dosage of one or both of the first and second active ingredient, and especially a strong synergism the active ingredients.

The molar ratio of FLT-3 inhibitor/an antisense oligonucleotide or a mel-1-specific RNAi construct in the combination is generally from 1/10 to 10/1, preferably from 1/5 to 5/1, e.g. 1/2, 1/1, 2/1, or 3/1.

### EXAMPLE 1

Materials and Methods

Reagents

Imatinib (STI571), PKC41228, and nilotinib(4-methyl-3-[[4-(3-pyridinyl)-2-pyrimidinyl]amino]-N-[5-(4-methyl-1H-imidazol-1-yl)-3-(trifluoromethyl)phenyl]benzamide)29 are provided by Novartis Pharma AG (Basel, Switzerland). Stock solutions of PKC412 and 4-methyl-3-[[4-(3-pyridinyl)-2-pyrimidinyl]amino]-[5-(4-methyl-1H-imidazol-1-yl)-3-(trifluoromethyl)phenyl]benzamide are prepared by dissolving in dimethyl-sulfoxide (DMSO; Merck, Darmstadt, Germany). Interleukin-4 (IL-4) is purchased from Peprotech (Rocky Hill, N.J.), RPMI 1640 medium and fetal calf serum (FCS) from PAA laboratories (Parching, Austria), L-glutamine and Iscove's modified Dulbecco's medium (IMDM) from Gibco Life Technologies (Gaithersburg, MD), ³H-thymidine from Amersham (Aylesbury, UK), and claribine (2-chlorodeoxyadenosine, 2CdA) from Janssen-Cilag (Beerse, Belgium). Antisense oligonucleotides (ASO; designed according to recently published sequences) are obtained from VBC Genomics (Vienna, Austria) or ISIS Pharmaceuticals (Carlsbad, Calif.), and siRNA from Dharmacon (Lafayette, Colo.).

Patient Characteristics and Purification of Neoplastic Mast Cells

A total number of 25 patients with systemic mastoycytosis (indolent systemic mastocytosis, ISM, n=17; aggressive systemic mastocytosis, ASM, n=3; mast cell leukemia, MCL, n=2; smouldering systemic mastocytosis, SSM, n=2; mast cell sarcoma, MCS, n=1) are examined. Bone marrow (BM) cells are obtained from the posterior iliac crest and collected in syringes containing preservative-free heparin. In each case, informed consent is obtained before biopsy. Diagnoses are established according to WHO criteria. In two patients with mast cell leukemia (MCL) and one with mast cell sarcoma (MCS), primary neoplastic MC are purified to homogeneity (purity >98%) using the PE-labeled mAb YB5.B8 (Pharmingen, San Diego, Calif.) and a FACS-Vantage cell sorter (Becton Dickinson, San Jose, Calif.) as described. All experiments are conducted with approval by the institutional review board and in accordance with the declaration of Helsinki.

Culture of HMC-1 Cells Exhibiting or Lacking KIT D816V

The human mast cell line HMC-1 generated from leukemic cells in a patient with MCL48 are obtained (Mayo Clinic, Rochester, Minn.). Two subclones of HMC-1 are used, namely HMC-1.1 harboring the KIT mutation G560V but not D816V, and HMC-1.2 cells exhibiting both the G560V mutation and D816V mutation of KIT.32 HMC-1 cells are grown in IMDM medium supplemented with 10% FCS, L-glutamine, and antibiotics at 37° C. and 5% CO₂. HMC-1 cells are re-thawed from an original stock every 4 to 8 weeks and passaged weekly. As control of ‘phenotypic stability’, HMC-1 cells are periodically checked for expression of KIT and the down-modulating effect of IL-4 (100 U/ml, 48 hours).

Treatment with Inhibitors

HMC-1 cells are incubated with various concentrations of the KIT D816V-targeting TK inhibitor PKC412 (100 pM through 10 μM), various concentrations of 4-methyl-3-[[4-(3-pyridinyl)-2-pyrimidinyl]amino]-N-[5-(4-methyl-1H-imidazol-1-yl)-3-(trifluoromethyl)phenyl]benzamide (1 nM through 100 μM), imatinib (3 nM through 300 μM), 2CdA (0.1-10,000 ng/mL), or control medium at 37° C. and 5% CO₂ for up to 48 hours. In a separate set of experiments, HMC-1 cells are transfected with mcl-1-specific ASO before being exposed to inhibitory drugs (see below). In other experiments, cells are transfected with various concentrations of Mcl-1-specific ASO (50 nM-250 nM) for up to 12 hours or with siRNA (200 nM) for 12 hours without further exposure to targeted drugs. After exposure to drugs or/and ASO, cells are subjected to ³H-thymidine uptake experiments, analysis of cell viability and apoptosis, or Western blotting.

Northern Blot Analysis and RT-PCR

Total RNA is isolated from HMC-1 cells using Trizol (Invitrogen, Carlsbad, Calif.) according to the manufacturers' instructions. RNA preparation and Northern blotting are performed essentially as described. In brief, 15 μg of total RNA are size-fractionated on 1.0% formaldehyde-agarose gels and transferred to nylon membranes (Hybond N, Amersham, Aylesbury, U.K.) as described. Hybridization is performed in rapid-hyb buffer (Amersham) using ³²P-labeled cDNAs specific for mcl-1 or β-actin. The mcl-1 probe is generated by excising the full length mcl-1 cDN from pBSK-Mcl-1 (a kind gift from Stanley J. Korsmeyer, Dana Farber Cancer Institute, Boston, Mass.) with EcoRI and XbaI (New England Biolabs, Beverly, Mass.) as described. Labeling is performed using the Megaprime-kit (Amersham). Blots are washed in 0.2×SSC (1×SSC=150 mM NaCl and 15 mM sodium citrate, pH 7.0) with 0.1% sodium dodecyl sulfate (SDS) for 1 hour at 42° C. for 2×30 minutes, and at 62° C. for another 30 minutes. Bound radioactivity is visualized by exposure to Biomax MS film (Kodak, Rochester, N.Y.) at −80° C. using intensifying screens (Kodak). mRNA expression levels are quantified by densitometry of autoradiograms using the E.A.S.Y. Win32 software (Herolab, Wiesloch, Germany). RT-PCR reactions are performed using the Protoscript First Strand cDNA Synthesis kit (New England Biolabs, Beverly, Mass.) using 1 μg RNA in a 50 μL reaction volume. PCR conditions are as follows: initial denaturation at 94° C. for 60 seconds, annealing at 55° C. for 60 seconds, polymerization at 72° C. for 60 seconds (35 cycles), and terminal extension at 72° C. for 10 minutes. The following primer pairs are used: mcl-1: 5′-TGC TGG AGT TGG TCG GGG AA-3′ (forward) (SEQ ID NO:3) and 5′-TCG TAA GGT CTC CAG CGC CT-3′ (reverse) (SEQ ID NO:4). β-actin: 5′-ATG GAT GAT GAT ATC GCC GCG-3′ (forward) (SEQ ID NO:5) and 5′-CTA GAA GCA TTT GCG GTG GAC GAT GGA GGG GCC-3′ (reverse) (SEQ ID NO:6). PCR products are resolved in 1% agarose gels containing 0.5 μg/mL ethidium bromide.

Western Blot Analysis

Western blot analysis is performed as described previously using lysates of HMC-1 cells and a polyclonal rabbit anti-human Mcl-1 antibody (Santa Cruz Biotechnology, Santa Cruz, Calif.). In brief, cells are lysed in phosphate buffered saline (PBS) containing 1% Triton-X-100 (Sigma, St. Louis, Mo.). Proteins are separated under reducing conditions by SDS-polyacrylamide gel electrophoresis and transferred to nitrocellulose membranes (Protran, Schleicher & Schuell, Dassel, Germany) in transfer buffer. Membranes are then exposed to anti-mouse/anti-rabbit IgG (BM Chemiluminescence Western blotting Kit, Roche). To confirm equal loading, membranes are re-probed with an anti-β-Actin antibody (Sigma). Chemiluminescence is detected by exposure to a Biomax MS film (Kodak).

Immunohistochemistry and Immunocytochemistry

In all patients with SM, expression of Mcl-1 in neoplastic BM MC is examined on serial sections (2 μm) prepared from paraffin-embedded, formalin-fixed BM specimens using the indirect immunoperoxidase staining technique. Endogenous peroxidase is blocked by CH₃OH/H₂O₂. Prior to staining with a polyclonal anti-Mcl-1 antibody (Santa Cruz) (work dilution: 1:50), BM sections are pretreated by microwave oven. Serial BM sections are incubated with the anti-Mcl-1 antibody (overnight) and the anti-tryptase antibody G3 (work dilution: 1:5,000 for one hour at room temperature) (Chemicon, Temecula, Calif.). Antibodies are diluted in 0.05 M Tris-buffered saline (TBS, pH 7.5) and 1% bovine serum albumin BSA (Sigma). After washing, slides are incubated with biotinylated goat anti-rabbit or horse anti-mouse IgG for 30 minutes, washed, and exposed to streptavidin-biotin-peroxidase complex for 30 minutes. 3-amino-9-ethyl-carbazole (AEC) is used as chromogen. Slides are counterstained in Mayer's Hemalaun. Immunocytochemistry is performed on cytospin preparations of HMC-1 cells as described using anti-Mcl-1 antibody (work dilution 1:200) and biotinylated goat anti-rabbit IgG (Biocarta, San Diego, Calif.). In select experiments, an Mcl-1-blocking peptide (Santa Cruz) is applied (work dilution 1:40). As chromogen, alkaline phosphatase complex (Biocarta) is used. Antibody-reactivity is made visible using Neofuchsin (Nichirei, Tokyo, Japan).

Transfection with mcl-1 Antisense Oligonucleotides (ASO) and siRNA

HMC-1.1 and HMC-1.2 cells are transfected with an mcl-1-specific 2′-O-methoxyethyl/2′-deoxynucleotide chimeric phosphorothioate ASO (5′-TTG GCT TTG TGT CCT TGG CG-3′) (SEQ ID NO:7) or with a scramble control oligonucleotide pool. Scramble control represents a mixture of A (adenine), G (guanine), T (thymine), and C (cytosine) bases, the resulting preparation containing an equimolar mixture of oligonucleotides. In a separate set of experiments, an annealed, purified, and desalted double-stranded mcl-1 siRNA (AAG AAA CGC GGU AAU CGG ACU) (SEQ ID NO:8) and AGU CCG AUU ACC GCG UUU CUU 3′ (mcl1, SEQ ID NO:9)) and a control siRNA against luciferase (CUU ACG CUG AGU ACU UCG A) (SEQ ID NO:10), both obtained from Dharmacon (Lafayette, Colo.), are applied. For transfection, 800,000 cells are seeded in 75 cm² culture plates at 37° C. for 24 hours. Mcl-1-ASO and Mcl-1 siRNA are complexed with Lipofectin-Reagent (Invitrogen) in RPMI 1640 medium essentially as described. In brief, HMC-1.1 or HMC-1.2 cells are incubated with various concentrations of mcl-1 ASO (50-250 nM) or 200 nM mcl-1-specific siRNA at 37° C. for 4 hours. After exposure to ASO, cells are washed and cultured in RPMI 1640 medium with 10% FCS in the presence or absence of various concentrations of TK inhibitors for another 12 hours before being analyzed. siRNA-treated cells are kept in control medium for 12 hours (without TK inhibitors) before being examined for the percentage of apoptotic cells and Western blotting.

³H-Thymidine Incorporation Assay and Determination of Cell Viability

To investigate the antiproliferative effects of Mcl-1 ASO, PKC412, 4-methyl-3-[[4-(3-pyridinyl)-2-pyrimidinyl]amino]-N-[5-(4-methyl-1H-imidazol-1-yl)-3-(trifluoromethyl)phenyl]benzamide, imatinib, and 2CdA, 3H-thymidine incorporation experiments are conducted using HMC-1.1 cells and HMC-1.2 cells. TK-inhibitors and 2CdA are applied to untreated cells or to cells transfected with ASO or universal (scramble) control. In particular, 4 hours after transfection with Mcl-1 ASO or control oligonucleotides, cells are cultured in 96-well microtiter plates (5×10⁴ cells per well) in the absence or presence of various concentrations of PKC412 (100 pM through 10 μM), 4-methyl-3-[[4-(3-pyridinyl)-2-pyrimidinyl]amino]-N-[5-(4-methyl-1H-imidazol-1-yl)-3-(trifluoromethyl)phenyl]benzamide (1 nM through 100 μM), imatinib (3 nM through 300 μM), or cladribine (2CdA; HMC-1.1: 0.1-10,000 ng/mL; HMC-1.2: 0.5-500 ng/mL), for 24 hours. Thereafter, 1 μCi ³H-thymidine is added to each well. Sixteen hours later, cells are harvested on filter membranes (Packard Bioscience, Meriden, Conn.) in a Filtermate 196 harvester (Packard Bioscience). Filters are then air-dried, and the bound radioactivity is measured in a β-counter (Top-Count NXT, Packard Bioscience). All experiments are performed in triplicates. Cell viability is determined by the trypan blue exclusion test. The percentage of apoptotic cells is determined on Wright-Giemsa-stained cytospin slides employing conventional cytomorphologic criteria.

Statistical Analysis

To determine the level of significance in differences found in the evaluation of data, the paired student t-test is applied. Results are considered to be significantly different with p<0.05. To determine synergistic effects of mcl-1 ASO and targeted drugs, combination index values are calculated according to published guidelines using commercially available software (CalcuSyn; Biosoft, Ferguson, Mo.).

Results

Detection of the Mcl-1 Protein in Neoplastic Mast Cells

METHOD: As assessed by immunohistochemistry and serial section staining, neoplastic MC are found to react with an antibody against Mcl-1 in all SM patients examined (n=25). Expression of Mcl-1 in neoplastic human mast cells is analyzed by immunohistochemical detection of tryptase and Mcl-1 in neoplastic MC in a patient with indolent SM (ISM). Adjacent bone marrow sections are incubated with antibodies against tryptase or Mcl-1. Immunohistochemistry is performed as described.

Immuncytochemical detection of Mcl-1 expression is further analyzed in HMC-1.2 cells exhibiting the KIT mutation D816V. Immunocytochemistry is performed using a polyclonal anti-Mcl-1 antibody. Preincubation of the antibody with a specific blocking peptide resulted in a negative stain. The same staining results are obtained with HMC-1.1 cells lacking KIT D816V. Northern blot analysis of HMC-1.1 cells and HMC-1.2 cells using an mcl-1-specific cDNA probe and a β-actin loading control. RT-PCR analysis of mcl-1 mRNA expression in K562 cells, HMC-1.1 cells, HMC-1.2 cells, as well as purified (purity >98%) neoplastic human mast cells is obtained from one patient with mast cell sarcoma (MCS) (#1) and two patients (#2 and #3) with mast cell leukemia (MCL). The PCR-reaction is controlled by ommitting the RT step (-RT).

RESULTS: Co-expression of tryptase and of Mcl-1 is observed in spindle-shaped neoplastic BM MC in a patient with ISM. Virtually all neoplastic MC in these infiltrates are found to stain positive for Mcl-1. There are also no differences in Mcl-1 expression (with regard to the percentage of stained MC in the infiltrates or the intensity of staining) in MC by immunohistochemistry when comparing patients in various categories of SM. In particular, Mcl-1 is found to be expressed in neoplastic MC in patients with ISM and SSM, as well as in high grade malignancies such as ASM and MCL. The human MCL-derived leukemia cell line HMC-1 (HMC-1.1 and HMC-1.2) is also found to express the Mcl-1 protein as determined by immunocytochemistry. The reactivity of the anti-Mcl-1 antibody with HMC-1.1 cells after preincubation with control medium or an WM-specific blocking peptide is performed, and the same result is obtained with HMC-1.2 cells.

Detection of mcl-1 mRNA in Neoplastic Mast Cells

In a next step, we examine expression of mcl-1 mRNA in primary neoplastic MC and HMC-1 cells. As assessed by Northern blotting, both HMC-1.1 cells and HMC-1.2 cells are found to express mcl-1 mRNA. Expression of mcl-1 mRNA in HMC-1 cells is confirmed by RT-PCR analysis. Moreover, we are able to demonstrate expression of mcl-1 mRNA in highly purified primary neoplastic MC in patients with MCL or MCS, by RT-PCR analysis.

Downregulation of Mcl-1 Expression Counteracts Viability of Neoplastic MC

METHOD: Effects of mcl-1 antisense oligonucleotides on Mcl-1 protein expression and cell viability in HMC-1.1 and HMC-1.2 cells are analyzed. HMC-1.1 cells and HMC-1.2 cells harbouring KIT D816V are transfected with an mcl-1 antisense oligonucleotide at 250 nM, a scramble control, or are left untransfected for 12 hours before being analyzed. Western Blot analysis of Mcl-1 expression is performed using an anti-Mcl-1 antibody. β-Actin served as loading control. Evaluation of non-viable (trypan blue-positive) cells is expressed as percentage of all nucleated cells. Numbers (%) of apoptotic cells. Results represent the mean±S.D. from 3 independent experiments.

Dose- and time-dependent effects of mcl-1 antisense oligonucleotides on neoplastic mast cells are analyzed via Western blot analysis of Mcl-1 expression in HMC-1.1 cells and HMC-1.2 cells after exposure to various concentrations of mcl-1 antisense oligonucleotides (50-250 nM) or Control medium for 12 hours. β-Actin serves as loading control. Time-dependent effects of mcl-1 antisense oligonucleotides (250 nM) and a scramble Control (250 nM) on expression of the Mcl-1 protein in HMC-1.1 cells and HMC-1.2 cells is observed. Mcl-1 expression is determined by Western blotting with β-Actin serving as a loading control. The time-dependent effects of the mcl-1 antisense oligonucleotides (250 nM) and of the scramble Control (250 nM) on cell viablity, i.e. the percentage of apoptotic HMC-1.1 cells and HMC-1.2 cells are obtained. Results represent the mean±S.D. from 3 independent experiments.

RESULTS: To investigate the role of Mcl-1 as a survival molecule and potential target in SM, Mcl-1 is knocked down in HMC-1.1 cells as well as in HMC-1.2 cells by an antisense oligonucleotide (ASO) approach. As determined by Western blotting, transfection of both cell lines with mcl-1 ASO (250 nM) virtually abolished expression of the Mcl-1 protein when comparing to the scramble control or to non-transfected cells. As expected, transfection with mcl-1 ASO leads to a substantial increase in trypan blue-positive cells (Control is about 1-5%; Scramble Control is about 5-8%; ASO is about 35-50%) and to a similarly substantial increase in the percentage of apoptotic cells in both HMC-1 subclones tested, namely HMC-1.1 cells and HMC-1.2 cells. The effects of the mcl-1 ASO on growth and survival of neoplastic MC (HMC-1) are found to be dose- and time-dependent. These data show that targeting of mcl-1 in neoplastic MC by an ASO approach is associated with loss of cell viability and induction of apoptosis.

Effects of mcl-1 siRNA on Neoplastic Mast Cells

METHOD: [001] Effects of mcl-1-siRNA are analyzed in neoplastic mast cells on expression of Mcl-1 protein and cell viability in HMC-1.1 cells and HMC-1.2 cells. Cells are left untransfected (Control) or are transfected with either an mcl-1-specific siRNA (mcl-1-siRNA; 200 nM) or a luciferase-specific (control)-siRNA (luc-siRNA; 200 nM) as described herein. Mcl-1 protein expression is determined by Western blotting using a polyclonal anti-Mcl -1 antibody. Equal loading is confirmed by probing for β-Actin. Cell viability is analyzed by recording the percentage (%) of apoptotic cells. Results represent the mean±S.D. from 3 independent experiments in each set of experiments.

RESULTS: In a next step, we apply an mcl-1-specific siRNA to further demonstrate the role of mcl-1 as a survival-related molecule and potential target in neoplastic MC. In these experiments, the mcl-1 siRNA is found to downregulate expression of the Mcl-1 protein in HMC-1.1 cells and HMC-1.2 cells. The siRNA induced down-regulation of Mcl-1 is found to be associated with an increase in apoptotic cells in HMC-1.1 cells as well as in HMC-1.2 cells. These data provide further evidence that mcl-1 is an essential survival factor in neoplastic MC.

Effects of Anti-Neoplastic Agents and Targeted Drugs on Expression of Mcl-1 in Neoplastic Mast Cells

METHOD: Effects of PKC412, nilotinib (i.e., 4-methyl-3-[[4-(3-pyridinyl)-2-pyrimidinyl]amino]-N-[5-(4-methyl-1H-imidazol-1-yl)-3-(trifluoromethyl)phenyl]benzamide)imatinib (i.e., GLIVEC or GLEEVEC) or 2CdA on expression of the Mcl -1 protein and on cell viability in HMC-1.1 cells lacking KIT D816V and in HMC-1.2 cells exhibiting KIT D816V is observed. Mcl-1 protein expression is determined by Western blotting using a polyclonal anti-Mcl-1 antibody. Equal loading is confirmed by probing for β-Actin. Cell viability is analyzed by recording the percentage (%) of apoptotic cells. Results documenting drug effects on cell viability represent the mean±S.D. from 3 independent experiments.

RESULTS: In a next step, we explore the effects of various targeted drugs on expression of Mcl-1 in HMC-1 cells. In these experiments we find that PKC412 (0.3-10 μM), 4-methyl-3-[[4-(3-pyridinyl)-2-pyrimidinyl]amino]-N-[5-(4-methyl-1H-imidazol-1-yl)-3-(trifluoromethyl)phenyl]benzamide (10-300 nM), and imatinib (30-300 nM), but not 2CdA (up to 1,000 ng/ml), down-regulate expression of the Mcl-1 protein in HMC-1.1 cells lacking KIT D816V. When applying the same drugs (at same concentrations) on HMC-1.2 cells exhibiting KIT D816V, we find that PKC412 (1-10 μM) and 2CdA (10-100 ng/ml) decrease the expression of Mcl-1 compared to control, whereas no significant effects are seen with either 4-methyl-3-[[4-(3-pyridinyl)-2-pyrimidinyl]amino]-N-[5-(4-methyl-1H-imidazol-1-yl)-3-(trifluoromethyl)phenyl]benzamide or imatinib over the dose range tested. In both cell lines, the drug-induced decrease in expression of the Mcl-1 protein is found to be accompanied by an increase in apoptotic cells.

Mcl-1 ASO cooperate with PKC412, 4-methyl-3-[[4-(3-pyridinyl)-2-pyrimidinyl]amino]-N-[5-(4-methyl-1H-imidazol-1-yl)-3-(trifluoromethyl)phenyl]benzamide(nilotinib), and imatinib in producing growth inhibition in neoplastic MC.

Based on the striking effects of the targeted drugs and of the mcl-1 specific ASO, we are interested to learn whether combinations of drugs and ASO would result in synergistic antiproliferative (or apoptosis-inducing) effects on HMC-1.1 cells and HMC-1.2 cells. In a first step, IC50 values are determined for each single drug in ³H-thymidine uptake experiments and experiments determining the number of apoptotic cells after exposure to drugs. The respective results (IC50 values) are summarized in Table 1. With regard to targeted drugs, the data are largely consistent to our previous observations. The concentrations of targeted drugs required to block proliferation are usually lower compared to those required for induction of apoptosis. In line with our previous data, PKC412 and 2CdA, and to a lesser degree 4-methyl-3-[[4-(3-pyridinyl)-2-pyrimidinyl]amino]-N-[5-(4-methyl-1H-imidazol-1-yl)-3-(trifluoromethyl)phenyl]benzamide are found to counteract cell growth in HMC-1.2 cells at pharmacologically relevant concentrations. By contrast, in HMC-1.1 cells, PKC412, 4-methyl-3-[[4-(3-pyridinyl)-2-pyrimidinyl]amino]-N-[5-(4-methyl-1H-imidazol-1-yl)-3-(trifluoromethyl)phenyl]benzamide, and imatinib are found to reduce cell growth and to induce apoptosis, whereas no significant effect is seen with 2CdA. The mcl-1 ASO is found to inhibit the proliferation and to induce apoptosis in both the HMC-1.1 and the HMC-1.2 subclone, with slightly higher IC50 values seen in HMC-1.2 cells.

TABLE 1
Effects of various targeted drugs on ³H-thymidine uptake (IC₅₀)
and induction of apoptosis (EC₅₀) in HMC-1.1 cells and HMC-1.2 cells

| | ³H-thymidine uptake (IC₅₀)* | | | | apoptosis (EC₅₀)* | | | |
|---|---|---|---|---|---|---|---|---|
| compound | HMC-1.1 | | HMC-1.2 | | HMC-1.1 | | HMC-1.2 | |
| mcl-1 ASO | 40-80 | nM | 60-100 | nM | 250 | nM | >250 | nM |
| PKC412 | 50-250 | nM | 50-250 | nM | 0.5-3 | μM | 0.5-3 | μM |
| nilotinib** | 3-10 | nM | 1-3 | μM | 30-80 | nM | >10 | μM |
| Imatinib | 10-30 | nM | >3 | μM | 50-150 | nM | >10 | μM |
| 2CdA | 0.1-0.3 | μg/mL | 5-20 | ng/mL | >1 | μg/mL | >1 | μg/mL |

*³H-thymidine uptake is analyzed after 24 hours of incubation with drugs or transfection with ASO, and the percentage of apoptotic cells determined on cytospin preparations after cells had been incubated with drugs for 24 hours. For evaluation of apoptosis, cells are examined 12 hours after transfection with ASO, because after 24 hours, the number of apoptotic cells is >50% in all instances.
**nilotinib is 4-methyl-3-[[4-(3-pyridinyl)-2-pyrimidinyl]amino]-N-[5-(4-methyl-1H-imidazol-1-yl)-3-(trifluoromethyl)phenyl] benzamide

Effects of mcl-1 antisense oligonucleotides and targeted drugs on growth and viability of neoplastic mast cells lacking (HMC-1.1) or exhibiting (HMC-1.2) KIT D816V are analyzed.

METHOD: Effects of various concentrations of PKC412, 4-methyl-3-[[4-(3-pyridinyl)-2-pyrimidinyl]amino]-N-5-(4-methyl-1H-imidazol-1-yl)-3-(trifluoromethyl)phenyl]benzamide(nilotinib), STI571 (imatinib), or 2CdA on ³H-thymidine uptake by HMC-1.1 cells transfected with a scramble control or with mcl-1 antisense oligonucleotides, 50 nM is performed. Results are expressed as percent of control (=scramble control) without targeted drug and represent the mean±SD of 3 independent experiments are analyzed. Effects of mcl-1 antisense oligonucleotides or scramble control (each 100 nM) applied with PKC412, nilotinib, STI571 (imatinib), or 2CdA or without the respective drug on cell viability, that is, percentage (%) of apoptotic cells are analyzed. Results represent the mean±SD of 3 independent experiments. Using CalcuSyn software, analyses of dose-effect relationships of PKC412, nilotinib, STI571 (imatinib), or 2CdA and mcl-1 antisense-induced apoptosis in HMC-1.1 cells are calculated according to the median effect method of Chou and Talalay. A combination index (CI) less than 1 indicates synergism.

Effects of various concentrations of PKC412, nilotinib, STI571 (imatinib), or 2CdA on ³H-thymidine uptake by HMC-1.2 cells transfected with a scramble control or mcl-1 antisense oligonucleotides (80 nM) are analyzed. Results are expressed as percent of control i.e. scramble control without targeted drug and represent the mean±SD of 3 independent experiments. Effects of mcl-1 antisense oligonucleotides or scramble control (each 100 nM) applied with PKC412, nilotinib, STI571 (imatinib), or 2CdA or without the respective drug on cell viability, that is, percentage (%) of apoptotic cells are analyzed. Results represent the mean±SD of 3 independent experiments. Using CalcuSyn software, analyses of dose-effect relationships of PKC412, nilotinib, STI571 (imatinib), or 2CdA and mcl-1 antisense-induced apoptosis in HMC-1.2 cells are calculated according to the median effect method of Chou and Talalay. A combination index (CI) less than 1 indicates synergism.

RESULTS: When comparing drug-induced responses of mcl-1-ASO-transfected HMC-1 cells with that of HMC-1 cells transfected with scramble control, substantial differences are found. In fact, in most cases, transfection with mcl-1 ASO is found to sensitize HMC-1 cells against TK inhibitors as well as against 2CdA. Interestingly, in HMC-1.1 cells, most combinations are found to be synergistic in nature. By contrast, in HMC-1.2 cells, synergistic effects are only seen with combinations of ASO and PKC412, and ASO and 2CdA, but not with ASO and 4-methyl-3-[[4-(3-pyridinyl)-2-pyrimidinyl]amino]-N-[5-(4-methyl-1H-imidazol-1-yl)-3-(trifluoromethyl)phenyl]benzamide or ASO and imatinib. All in all, these data show that Mcl-1 can be employed as a new drug target in neoplastic MC and that an mel-1-specific ASO is capable of sensitizing HMC-1 cells against 2CdA and novel TK inhibitors.

Discussion

Mcl -1 is a well characterized member of the Bcl-2 family that has recently been implicated in the pathogenesis of various myeloid neoplasms. Systemic mastocytosis (SM) is a myeloid neoplasm characterized by abnormal growth and accumulation of MC in visceral organs. We provide evidence that neoplastic MC in patients with SM as well as the human mast cell leukemia cell line HMC-1 express Mcl-1 in a constitutive manner. Moreover, our data show that Mcl-1 is a critical survival-molecule in neoplastic MC, and that targeting of Mcl-1 by ASO or siRNA is associated with decreased survival of neoplastic MC and with an increased response to KIT tyrosine kinase inhibitors, including PKC412 or 4-methyl-3-[[4-(3-pyridinyl)-2-pyrimidinyl]amino]-N-[5-(4-methyl-1H-imidazol-1-yl)-3-(trifluoromethyl)phenyl]benzamide.

Normal and neoplastic MC are extremely long-lived cells compared to most other myeloid lineages including basophils, but the mechanisms and survival factors responsible for their long term survival remain largely unknown. Although members of the Bcl-2 family have been implicated as essential survival factors in granulomonocytic cells, little is known about the expression and role of these molecules in MC. So far, it has been reported that neoplastic MC as well as HMC-1 cells express Bcl-2. In addition, neoplastic MC in SM are described to display Bcl-xL. In this study, we are able to demonstrate the expression of Mcl-1 in neoplastic MC at the mRNA- and protein level, as well as by functional analyses employing specific ASO and siRNA. Thus, based on our data, Mcl-1 must be regarded as a crucial survival factor expressed in neoplastic MC.

The clinical course in SM is variable and ranges from asymptomatic with low proliferative capacity of MC and normal life expectancy to highly aggressive cases with rapid proliferation of neoplastic MC and a short survival. We are therefore interested to learn whether the levels of Mcl-1 would vary among the groups of patients with SM. However, no apparent differences in Mcl-1 expression in MC are detected when comparing low- and high-grade MC malignancies. This observation can be explained by the fact that Mcl-1 expression is related to survival of MC, rather than to their proliferative capacity. In fact, even in indolent SM, MC exhibit extremely long survival. On the other hand, Cervero et al. reported an increased expression of Bcl-2 in BM MC in patients with MCL, compared to ISM using a highly sensitive flow cytometry technique. Therefore, we cannot exclude that MC in MCL express slightly higher levels of Mcl-1 compared to normal MC or MC in ISM, as the technique applied in our study is less sensitive compared to flow cytometry.

Little is known about the regulation of expression of Mcl-1 in neoplastic myeloid cells. In chronic myeloid leukemia (CML), the disease-related oncoprotein BCR/ABL is found to promote expression of Mcl-1.46 In SM, the D816V-mutated variant of KIT serves as a disease-related oncoprotein in a similar manner as compared to BCR/ABL in CML. To explore whether this oncoprotein could be involved in the regulation of expression of Mcl-1 in neoplastic MC, we utilized inhibitors of the TK activity of KIT. In these experiments, PKC412, 4-methyl-3-[[4-(3-pyridinyl)-2-pyrimidinyl]amino]-N-5-(4-methyl-1H-imidazol-1-yl)-3-(trifluoromethyl)phenyl]benzamide, and imatinib are found to downregulate expression of Mcl-1 in HMC-1.1 (lacking KIT D816V but expressing KIT G560V), whereas in HMC-1.2 cells expressing KIT D816V, only PKC412 and to a lesser degree 4-methyl-3-[[4-(3-pyridinyl)-2-pyrimidinyl]amino]-N-[5-(4-methyl-1H-imidazol-1-yl)-3-(trifluoromethyl)phenyl]benzamide, decreased the expression of the Mcl -1 protein. Since PKC412 potently inhibits of the TK activity of KIT D816V, and since this mutation confers resistance against imatinib, these observations could be construed to favor the hypothesis that KIT D816V contributes to abnormal expression of Mcl-1 in neoplastic MC. On the other hand, our data also suggest that Mcl-1 expression is detectable and KIT-dependent in HMC-1 cells lacking KIT D816V, as KIT TK inhibitors downregulated Mcl-1 expression in HMC-1.1 cells. This observation is of particular interest, since MC in high grade malignancies (ASM or MCL) often lack KIT D816V or have KIT D816V-negative sublcones that may be selected during TK-targeted therapy to cause relapse.

Mcl-1 is a well established survival molecule that counteracts apoptosis in myeloid cells. To provide evidence for the functional significance of Mcl-1 expression in neoplastic MC, HMC-1 cells are transfected with mcl-1-specific ASO or mcl-1-specific siRNA. In both HMC-1 subclones, the specific ASO- and siRNA-induced knock-down of the Mcl-1 protein is demonstrable by Western blotting and is accompanied by a significant increase in apoptotic cells. In addition, the proliferation of HMC-1 cells decreased after transfection with Mcl-1 ASO compared to cells transfected with a scramble control. These data provide solid evidence that Mcl-1 is an important survival factor in HMC-1 cells.

We next compared the proliferation-inhibiting and apoptosis-inducing effects of mcl-1 ASO and of various targeted drugs including 2CdA and novel KIT TK inhibitors. An interesting aspect in these experiments is that proliferation measured by ³H-thymidine uptake is suppressed at lower concentrations of TK inhibitors when compared to induction of apoptosis. This discrepancy may have significant implications when comparing effects of various inhibitory drugs, and may best be explained by the fact that uptake of ³H-thymidine is a more sensitive parameter of growth and may be affected before signs of apoptosis occur.

ASM and MCL are high grade MC malignancies with a grave prognosis and a short survival. In these patients, a number of different factors may contribute to abnormal growth and survival of MC, and it is difficult to identify drugs counteracting growth of neoplastic cells in these patients. However, during the past few years, a number of novel treatment concepts and targeted drugs have been identified which overcome drug-resistance in high grade MC neoplasms. One of these drugs is PKC412, a novel TK inhibitor that counteracts TK activity of mutants of Flt3, wild type KIT as well as KIT D816V. Correspondingly, PKC412 is found to counteract growth and Mcl-1 expression in HMC-1.2 cells expressing KIT D816V in the present study. With regard to growth inhibition, these data confirm previous observations by us and by others. However, even PKC412 as a single drug may not be able to induce durable complete remissions in patients with ASM or MCL.

A most attractive approach in TK-driven, drug-resistant myeloid neoplasms is to combine targeted drugs with each other or with conventional drugs. In case of ASM or MCL, it may thus be reasonable to consider combinations of drugs employing PKC412, other TK inhibitors, and other targeted drugs. The results of our study show that mcl-1 ASO and PKC412 as well as mcl-1 ASO and 4-methyl-3-[[4-(3-pyridinyl)-2-pyrimidinyl]amino]-N-[5-(4-methyl-1H-imidazol-1-yl)-3-(trifluoromethyl)phenyl]benzamide cooperate with each other in producing growth inhibition in HMC-1.1 cells and HMC-1.2 cells. In addition, cooperative effects are also seen when applying imatinib and mcl-1 ASO in HMC-1.1 cells. An interesting observation is that the cooperative drug effects between mcl-1 ASO and PKC412 are synergistic in both HMC-1 subclones, whereas the interactions between mcl-1 ASO and 4-methyl-3-[[4-(3-pyridinyl)-2-pyrimidinyl]amino]-N-[5-(4-methyl-1H-imidazol-1-yl)-3-(trifluoromethyl)phenyl]benzamide are only synergistic in HMC-1.1 cells, but not in HCM-1.2 cells. These differences are best explained by the less pronounced anti-proliferative effects of imatinib and 4-methyl-3-[[4-(3-pyridinyl)-2-pyrimidinyl]amino]-N-[5-(4-methyl-1H-imidazol-1-yl)-3-(trifluoromethyl)phenyl]benzamide compared to the effects of PKC412 on HMC-1.2 cells which display KIT D816V.

2CdA is a drug that has recently been introduced as a novel effective cyto-reductive agent for the treatment of advanced SM. In the current study, we found that 2CdA inhibits growth and Mcl-1 expression in HMC-1.2 cells much more potently compared to HMC-1.1 cells, which may be of great clinical importance, since most patients with SM including those with ASM and MCL, display this KIT mutation. Thus, 2CdA is the first drug described to act better in MC expressing KIT D816V as on MC lacking this KIT mutant. However, despite these effects, 2CdA did not produce synergistic anti-proliferative effects with mcl-1 ASO in the two HMC-1 subclones analyzed.

A number of clinical Phase I/II trials have attempted to employ ASO-type drugs. However, despite first encouraging results, some reservations have been made in the past concerning the possibility to apply such drugs in cancer patients. With regard to mcl-1 and SM, further preclinical and clinical studies will be required to define whether mel-1-targeting treatment concepts can be developed far enough to enter clinical trials in ASM and MCL.

In summary, our data show that neoplastic MC express Mcl-1, and that targeting of Mcl -1 is associated with decreased survival and increased responsiveness against TK inhibitors such as PKC412. These data suggest that Mcl-1 is a novel interesting target in neoplastic MC that may help to overcome resistance against TK inhibitors.

1. A method of treating myelodysplastic syndromes, lymphomas and leukemias, and solid tumors in a mammal which comprises treating the mammal in need of such treatment simultaneously, concurrently, separately or sequentially with pharmaceutically effective amounts of (a) a FLT-3 inhibitor, or a pharmaceutically acceptable salt or a prodrug thereof, and (b) an mcl-1 antisense oligonucleotide or a mcl-1-specific RNAi construct.

2. The method according to claim 1 for treating acute myeloid leukemia (AML).

3. The method according to claim 1, wherein the FLT-3 inhibitor is a staurosporine derivative or 4-methyl-3-[[4-(3-pyridinyl)-2-pyrimidinyl]amino]-N-[5-(4-methyl-1H-imidazol-1-yl)-3-(trifluoromethyl)phenyl]benzamide or imatinib.

4. The method according to claim 3, wherein the staurosporine derivative is selected from
the compounds of formula, 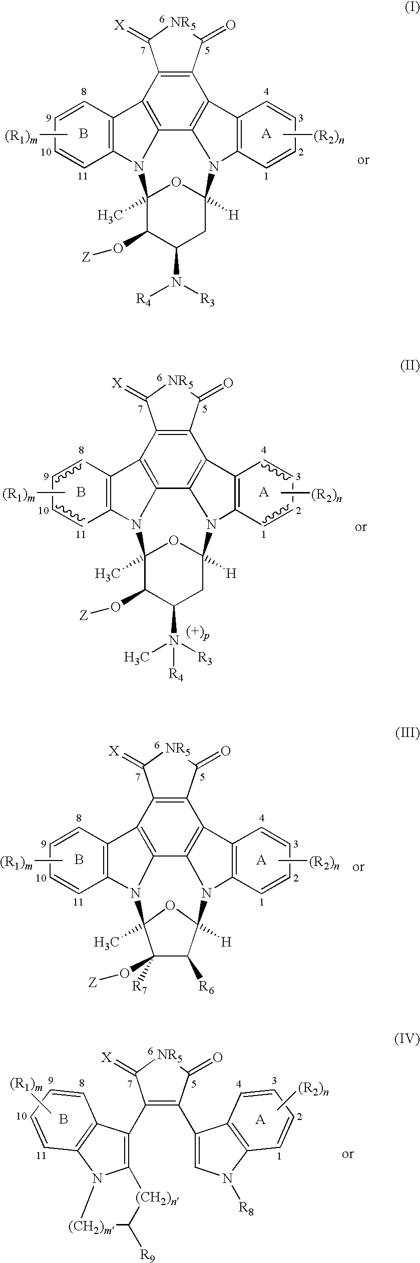 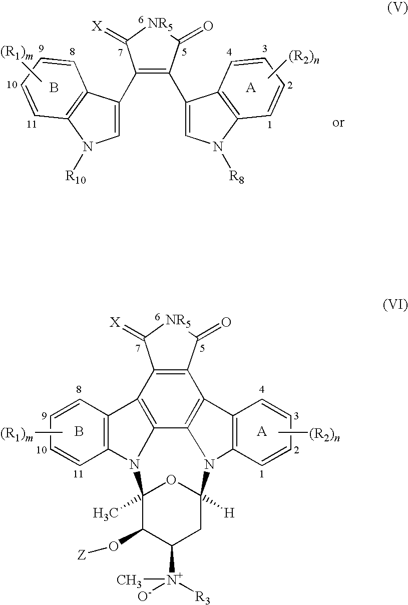
wherein R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 1 to and including 4;
R₃, R₄, R₈ and R₁₀ are, independently of one another, hydrogen, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent;
or R₃ is acyl with up to 30 carbon atoms and R₄ not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy,carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

5. The method according to claim 3, wherein the staurosporine derivative is a staurosporin derivative of formula I, 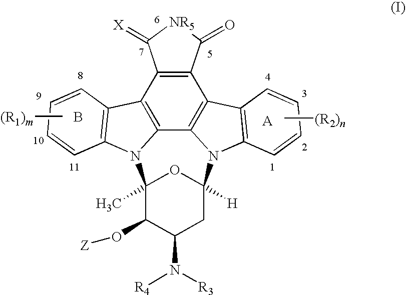
wherein
m and n are each 0;
R₃ and R₄ are independently of each other hydrogen,
lower alkyl unsubstituted or mono- or disubstituted, especially monosubstituted, by radicals selected independently of one another from carboxy; lower alkoxycarbonyl; and cyano;
or
R₄ is hydrogen or —CH₃, and
R₃ is acyl of the subformula R^{o} —CO, wherein R^{o} is lower alkyl; amino-lower alkyl, wherein the amino group is present in unprotected form or is protected by lower alkoxycarbonyl; tetrahydropyranyloxy-lower alkyl; phenyl; imidazolyl-lower alkoxyphenyl; carboxyphenyl; lower alkoxycarbonylphenyl; halogen-lower alkylphenyl; imidazol-1-ylphenyl; pyrrolidino-lower alkylphenyl; piperazino-lower alkylphenyl; (4-lower alkylpiperazinomethyl)phenyl; morpholino-lower alkylphenyl; piperazinocarbonylphenyl; or (4-lower alkylpiperazino)phenyl;
or is acyl_of the subformula R^{o} —O—CO—, wherein R^{o} is lower alkyl;
or is acyl of the subformula R^{o} HN—C(═W)—, wherein W is oxygen and R^{o} has the following meanings:
morpholino-lower alkyl, phenyl, lower alkoxyphenyl, carboxyphenyl, or lower alkoxycarbonylphenyl;
or R₃ is lower alkylphenylsulfonyl, typically 4-toluenesulfonyl;
R₅ is hydrogen or lower alkyl,
X stands for 2 hydrogen atoms or for O;
Z is methyl or hydrogen;
or a salt thereof, if at least one salt-forming group is present.

6. The method according to claim 3, wherein the staurosporine derivative is N-[(9S,10R,11R,13R)-2,3,10,11,12,13-hexahydro-10-methoxy-9-methyl-1-oxo-9,13-epoxy-1H,9H-formula diindolo[1,2,3-gh:3′,2′,1′-lm]pyrrolo[3,4-j][1,7]benzodiazonin-11-yl]-N-methylbenzamide of the (VII): 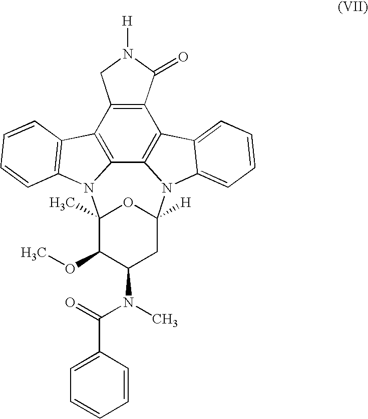
or a salt thereof.

7-12. (canceled)

13. A pharmaceutical composition comprising (a) a FLT-3 inhibitor and (b) an mcl-1 antisense oligonucleotide or a mel-1-specific RNAi construct for the treatment of myelodysplastic syndromes, lymphomas and leukemias and solid tumors.

14. The pharmaceutical composition according to claim 13 for treating acute myeloid leukemia (AML), colorectal cancer (CRC) or non-small cell lung cancer (NSCLC).

15. The pharmaceutical composition according to claim 13, wherein the FLT-3 inhibitor is -[(9S,10R,11R,13R)-2,3,10,11,12,13-hexahydro-10-methoxy-9-methyl-1-ox-9,13-epoxy-1H,9H-diindolo[1,2,3-gh:3′,2′,1′-lm]pyrrolo[3,4-j][1,7]benzodiazonin-11-yl]-N-methylbenzamide of the formula (VII): 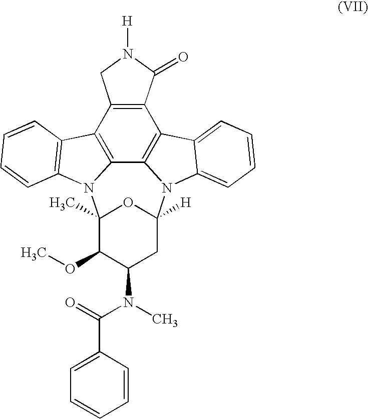
or a salt thereof and an mcl-1 antisense oligonucleotide or a mcl-1-specific RNAi construct.

16. An isolated nucleic acid comprising a nucleotide sequence selected from SEQ ID NO:3, SEQ ID NO:4, SEQ ID NO:7, SEQ ID NO:8 and SEQ ID NO:9, or its complementary strand sequence.

17. A vector comprising the isolated nucleic acid of claim 16.

18. The vector of claim 17, wherein the vector is an expression vector.

19. A host cell comprising the isolated nucleic acid of claim 16.

20. A short hairpin RNA (shRNA) comprising a sequence selected from SEQ ID NO:7, SEQ ID NO:8 and SEQ ID NO:9 or its complementary strand sequence.

21. An antisense oligonucleotide comprising a sequence selected from SEQ ID NO:3 and SEQ ID NO:4 or its complementary strand sequence.
